(19) United States
(12) Patent Application Publication
(10) Pub. No.: US 2024/0286172 A1
(43) Pub. Date: Aug. 29, 2024

(54) ULTRASONIC TRANSDUCERS AND METHODS FOR PREPARING MATCHING LAYERS THEREOF

(71) Applicant: WUHAN UNITED IMAGING HEALTHCARE CO., LTD., Wuhan (CN)

(72) Inventors: Zheng CHENG, Wuhan (CN); Yu LIU, Wuhan (CN); Kang SI, Shanghai (CN)

(73) Assignee: WUHAN UNITED IMAGING HEALTHCARE CO., LTD., Wuhan (CN)

(21) Appl. No.: 18/660,216

(22) Filed: May 9, 2024

Related U.S. Application Data
(63) Continuation of application No. PCT/CN2022/135513, filed on Nov. 30, 2022.

(30) Foreign Application Priority Data
Nov. 30, 2021 (CN) 202111446507.6
Sep. 26, 2022 (CN) 202211173845.1

(51) Int. Cl. B06B 1/06

(52) U.S. Cl. CPC B06B 1/0618; B06B 1/0622; B06B 2201/76

(57) ABSTRACT

The present disclosure provides an ultrasonic transducer and a method for preparing a matching layer. The ultrasonic transducer may include a piezoelectric layer and a matching layer. The matching layer may be provided between the piezoelectric layer and an object to be measured, the piezoelectric layer may be acoustically matched with the object through the matching layer, and the piezoelectric layer may facilitate conversion between ultrasonic waves and electrical energy. A sound velocity in the matching layer may be in a gradient distribution in at least one direction. Through the gradient distribution of the sound velocity in the matching layer, ultrasonic waves of different frequencies may have the same wavelength. Thus, an acoustic matching between the piezoelectric layer of different structures and the human body tissues may be realized, which improves transmittance of the ultrasonic wave in the matching layer and increases a bandwidth of the ultrasonic transducer.

### CROSS-REFERENCE TO RELATED APPLICATIONS

This application is a Continuation of International Application No. PCT/CN2022/135513, filed on Nov. 30, 2022, which claims priority to Chinese Patent Application No. 202111446507.6, filed on Nov. 30, 2021, and Chinese Patent Application No. 202211173845.1 filed on Sep. 26, 2022, the entire contents of each of which are hereby incorporated by reference.

### TECHNICAL FIELD

The present disclosure relates to the medical field, and in particular, to an ultrasonic transducer and a method for preparing a matching layer.

### BACKGROUND

Medical ultrasound imaging is a very important technology in modern medical imaging. The ultrasonic transducer, as a core component of a medical ultrasound imaging device, may convert electrical energy into ultrasonic waves through the piezoelectric effect and transmit the ultrasonic waves into the human body, and convert ultrasonic waves reflected by the human body into electrical signals. The electrical signals are processed to form a corresponding image, such as an ultrasound image.

The bandwidth is one of the important performance parameters for the ultrasonic transducer. In the prior art, ultrasonic transducers with higher bandwidth are often required to transmit and receive ultrasonic waves of different frequencies to meet the requirements of different scenarios, such as the requirement of harmonic imaging on the bandwidth.

Therefore, it is desirable to provide an ultrasonic transducer with a relatively high bandwidth, and a method for preparing a matching layer.

### SUMMARY

One embodiment of the present disclosure provides an ultrasonic transducer. The ultrasonic transducer may include a piezoelectric layer and a matching layer. The matching layer may be provided between the piezoelectric layer and an object to be measured. The piezoelectric layer may be acoustically matched with the object through the matching layer, and the piezoelectric layer may be configured to facilitate conversion between ultrasonic waves and electrical energy. A sound velocity in the matching layer may be in a gradient distribution in at least one direction. The present disclosure, through the gradient distribution of the sound velocity of the matching layer, makes ultrasonic waves of different frequencies have the same wavelengths in matching layers of different structures. Then, acoustic matching between piezoelectric layers of different structures and tissues of the human body may be realized. Therefore, transmittances of ultrasonic waves in the matching layer may be improved, and the bandwidth of the ultrasonic transducer may be increased.

In some embodiments, a thickness of the piezoelectric layer may include a first thickness and a second thickness. The first thickness may be unequal to the second thickness.

In some embodiments, the gradient distribution of the sound velocity in the matching layer may be associated with a thickness distribution of the piezoelectric layer.

In some embodiments, the gradient distribution of the sound velocity in the matching layer may be negatively correlated with the thickness distribution of the piezoelectric layer.

In some embodiments, the ultrasonic transducer may further include a backing layer. The backing layer may include a first impedance layer and a second impedance layer. The first impedance layer may be connected to the second impedance layer, and the first impedance layer may be connected to a surface of the piezoelectric layer away from the matching layer. An impedance of the first impedance layer may be higher than an impedance of the second impedance layer.

In some embodiments, the piezoelectric layer may be of equal thickness at different sites. A thickness of the first impedance layer may include a third thickness and a fourth thickness. The third thickness may be unequal to the fourth thickness.

In some embodiments, the gradient distribution of the sound velocity in the matching layer may be associated with a thickness distribution of the first impedance layer.

In some embodiments, the gradient distribution of the sound velocity in the matching layer may be negatively correlated with the thickness distribution of the first impedance layer.

In some embodiments, the matching layer may be of equal thickness at different sites.

In some embodiments, a thickness of the matching layer may include a fifth thickness and a sixth thickness. The fifth thickness may be unequal to the sixth thickness.

In some embodiments, the piezoelectric layer may include one or more piezoelectric array elements. The at least one direction may include a length direction and/or a thickness direction of the one or more piezoelectric array elements.

In some embodiments, the matching layer may include a first filler and a second filler. A first sound velocity corresponding to the first filler may be different from a second sound velocity corresponding to the second filler.

In some embodiments, the first filler and the second filler may be respectively filled at different positions in a same plane of the matching layer to achieve a gradient distribution of the sound velocity in the matching layer.

In some embodiments, acoustic impedances and/or thicknesses at positions of the matching layer at which the first filler and the second filler are respectively filled, may be the same.

In some embodiments, the first filler may include at least one type of material that is different from the second filler. The first filler and the second filler may include one or more same types of materials. Percentages, sizes, and/or structures of at least one of the one or more same types of materials in the first filler and the second filler may be different.

In some embodiments, the first filler and the second filler may include an inorganic filler and/or an organic filler.

In some embodiments, the first filler and/or the second filler may include an epoxy resin. The first filler and/or the second filler may further include at least one of a material with a sound velocity in a first sound velocity range, a material with a sound velocity in a second sound velocity range, a material with a density in a first density range, and a material with a density in a second density range. The first sound velocity range may be smaller than the second sound velocity range, and the first density range may be greater than the second density range.

In some embodiments, the first sound velocity range may be from 800 m/s to 2000 m/s, the second sound velocity range may be from 2800 m/s to 11000 m/s, the first density range may be from 3 g/cm³ to 20 g/cm³, and the second density range may be from 0.1 g/cm³ to 0.8 g/cm³.

In some embodiments, a content of the epoxy resin may be 100 g. A content of the material with the sound velocity in the first sound velocity range may be less than or equal to 60 g, a content of the material with the sound velocity in the second sound velocity range is less than or equal to 130 g, a content of the material with the density in the first density range is less than or equal to 500 g, or a content of the material with the density in the second density range is less than or equal to 20 g.

In some embodiments, the material with the sound velocity in the first sound velocity range may include rubber. The material with the sound velocity in the second sound velocity range may include a metal oxide and/or an inorganic non-metallic compound of a solid structure. The material with the density in the first density range may include a metal. The material with the density in the second density range may include an inorganic non-metallic compound of a hollow structure and/or a plastic expanding microsphere.

In some embodiments, materials of the first filler may include the epoxy resin, the rubber, and the metal. Materials of the second filler may include the epoxy resin, the metal oxide, and the inorganic non-metallic compound of hollow structure. A first sound velocity of the first filler may be less than a second sound velocity of the second filler. An acoustic impedance of the first filler and an acoustic impedance of the second filler may be the same.

In some embodiments, the epoxy resin may include at least one of an epoxy resin of a bisphenol A type or an epoxy resin of a bisphenol F type. the rubber may include at least one of thermoplastic SBS elastomer, nitrile rubber, a butyl rubber, styrene-butadiene rubber, male-butadiene rubber, ethylene propylene diene monomer (EPDM) rubber, silicone rubber, or fluorine rubber. The metal may include at least one of tungsten, copper, iron, or lead. The metal oxide may include at least one of a tungsten trioxide, an iron oxide, an aluminum trioxide, a zinc oxide, or a magnesium oxide. The inorganic non-metallic compound may include at least one of glass, ceramic, or boron carbide.

In some embodiments, a sound velocity of the first filler or a sound velocity of the second filler may be within a range from 1400 m/s to 3500 m/s.

In some embodiments, the first sound velocity of the first filler in the matching layer may be associated with a first thickness of the piezoelectric layer. The second sound velocity of the second filler in the matching layer may be associated with a second thickness of the piezoelectric layer. The first thickness may be greater than the second thickness. The first sound velocity may be less than the second sound velocity.

One of the embodiments of the present disclosure further provides a method for preparing a matching layer. The method may include preparing a first filler and a second filler according to a target acoustic impedance and a target gradient distribution of a sound velocity that need to be achieved in the matching layer; disposing the first filler and the second filler in corresponding predetermined positions, respectively; and obtaining the matching layer by curing at a predetermined temperature for a predetermined time.

In some embodiments, the predetermined temperature may be within a range from 20° C. to 100° C., and the predetermined time may be within a range from 2 h to 48 h.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures, and wherein:

FIG. 1 is a schematic diagram illustrating an exemplary structure of an ultrasonic transducer according to some embodiments of the present disclosure;

FIG. 2 is a graph illustrating exemplary frequency characteristic curves of an ultrasonic transducer according to some embodiments of the present disclosure;

FIG. 3 is a schematic diagram illustrating an exemplary three-dimensional structure of a first type of ultrasonic transducer according to some embodiments of the present disclosure;

FIG. 4 is a schematic diagram illustrating an exemplary structure of a first type of ultrasonic transducer according to some embodiments of the present disclosure;

FIG. 5 is a schematic diagram illustrating an exemplary structure of a first type of piezoelectric layer of an ultrasonic transducer according to some embodiments of the present disclosure;

FIG. 6 is a schematic diagram illustrating an exemplary structure of a second type of piezoelectric layer of an ultrasonic transducer according to some embodiments of the present disclosure;

FIG. 7 is a schematic diagram illustrating an exemplary structure of a third type of piezoelectric layer of an ultrasonic transducer according to some embodiments of the present disclosure;

FIG. 8 is a schematic diagram illustrating an exemplary structure of a fourth type of piezoelectric layer of an ultrasonic transducer according to some embodiments of the present disclosure;

FIG. 9 is a schematic diagram illustrating an exemplary structure of a fifth type of piezoelectric layer of an ultrasonic transducer according to some embodiments of the present disclosure;

FIG. 10 is a schematic diagram illustrating an exemplary structure of a sixth type of piezoelectric layer of an ultrasonic transducer according to some embodiments of the present disclosure;

FIG. 11 is a schematic diagram illustrating an exemplary structure of a second type of ultrasonic transducer according to some embodiments of the present disclosure;

FIG. 12 is a schematic diagram illustrating an exemplary three-dimensional structure of a second type of ultrasonic transducer according to some embodiments of the present disclosure;

FIG. 13 is a schematic diagram illustrating an exemplary cross sectional view in a Y-Z plane of a second type of ultrasonic transducer after bonding and cutting according to some embodiments of the present disclosure;

FIG. 14 is a schematic diagram illustrating an exemplary multi-array structure of a second type of ultrasonic transducer after bonding and cutting in an X-Y plane according to some embodiments of the present disclosure;

FIG. 15 is a schematic diagram illustrating an exemplary first type of backing structure of an ultrasonic transducer after bonding according to some embodiments of the present disclosure;

FIG. 16 is a schematic diagram illustrating an exemplary second type of backing structure of an ultrasonic transducer after bonding according to some embodiments of the present disclosure;

FIG. 17 is a schematic diagram illustrating an exemplary third type of backing structure of an ultrasonic transducer after bonding according to some embodiments of the present disclosure;

FIG. 18 is a schematic diagram illustrating an exemplary fourth type of backing structure of an ultrasonic transducer after bonding having a matching layer with a non-uniform thickness according to some embodiments of the present disclosure; and

FIG. 19 is a flowchart illustrating an exemplary process for preparing a matching layer according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system”, “device”, “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one”, “a”, “a kind” and/or “the” are not especially singular but may include the plural unless the context expressly suggests otherwise. In general, the terms “comprise,” “comprises,” “comprising,” “include,” “includes,” and/or “including,” merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

In some embodiments, a medical device may include an image processing device and an ultrasonic transducer. The ultrasound transducer may convert electrical energy into ultrasonic waves that may emit into the human body and convert ultrasonic waves (e.g., echo signal(s)) reflected by the human body into electrical signal(s). The echo signal(s) corresponding to the electrical signal(s) may reflect a state of internal organs of the human body. The image processing device may be connected to the ultrasonic transducer. The image processing device may receive the electrical signal(s) from the ultrasonic transducer and process the electrical signal(s) to obtain a corresponding image (e.g., an ultrasound image).

The image processing device may be a device for processing received signals to obtain an image. In some embodiments, the image processing device may include a plurality of processing units. The plurality of processing units may process the electrical signal(s) from the ultrasonic transducer to obtain a processed image.

The present disclosure provides an ultrasonic transducer. The ultrasonic transducer may use the piezoelectric effect to realize the conversion between the ultrasonic waves and the electrical energy, which may be applied to various scenarios using the ultrasonic waves for measurement. For example, the ultrasonic transducer may be applied to a medical imaging device for outputting medical images according to the ultrasonic waves returned by an object to be measured (e.g., human tissues, etc.). As another example, the ultrasonic transducer may be applied to an underwater monitoring device for obtaining underwater images based on ultrasonic waves returned by an underwater object.

In some embodiments, the ultrasonic transducer may include a piezoelectric layer. The piezoelectric layer may convert the electrical energy into the ultrasonic waves to be emitted to the object to be measured (e.g., human tissues, etc.). However, in some embodiments, there may be a significant difference between an acoustic impedance of the piezoelectric layer and an acoustic impedance of the object, causing most of the ultrasonic waves to be reflected, resulting in a relatively low transmittance of the ultrasonic waves from the ultrasonic transducer to the human tissues, affecting efficiency of the ultrasonic transducer to receive the ultrasonic waves of different frequencies, thus reducing a bandwidth of the ultrasonic transducer.

The ultrasonic transducer provided in the present disclosure includes a matching layer and a piezoelectric layer. The matching layer may be provided on an upper surface of the piezoelectric layer to acoustically match the object to be measured (e.g., human tissues, etc.). As the transmittance is affected by a thickness of the matching layer and wavelengths of the ultrasonic waves, a gradient distribution of a sound velocity in the matching layer may allow the ultrasonic waves of different frequencies to have the same wavelength in matching layers of different structures. In such cases, an acoustic matching between piezoelectric layers of different structures and human tissues may be realized, thereby increasing the transmittance of ultrasonic waves in the matching layer, and increasing the bandwidth of the ultrasonic transducer.

It should be understood that application scenarios of the ultrasonic transducer of the present disclosure are only some examples or embodiments of the present disclosure. Those having ordinary skills in the art, without further creative efforts, may apply the present disclosure to other similar scenarios according to these drawings.

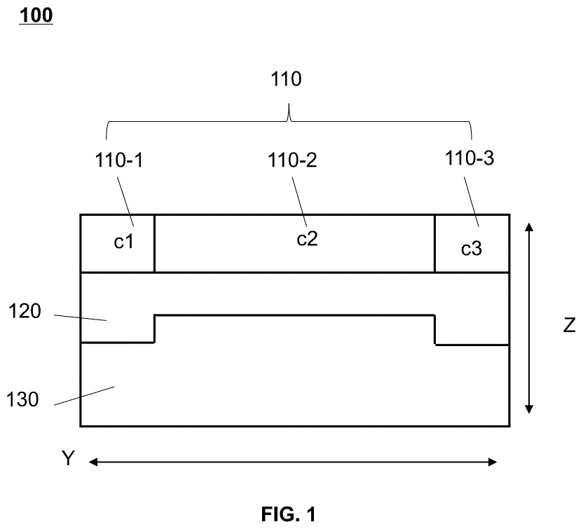

The ultrasonic transducer in the embodiments of the present disclosure is described in detail below in conjunction with FIGS. 1-10. It should be noted that the following embodiments are merely described to explain the present disclosure and do not constitute a limitation of the present disclosure.

FIG. 1 is a schematic diagram illustrating an exemplary structure of an ultrasonic transducer according to some embodiments of the present disclosure. In some embodiments, the ultrasonic transducer 100 may include a matching layer 110, and a piezoelectric layer 120. The matching layer 110 may be provided between the piezoelectric layer 120 and an object to be measured. The piezoelectric layer 120 may be acoustically matched with the object through the matching layer 110. The piezoelectric layer 120 may be configured to facilitate conversion between ultrasonic waves and electrical energy. A sound velocity in the matching layer may be in a gradient distribution in at least one direction.

The matching layer 110 may have a layered structure that couples (i.e., acoustically matches) acoustic impedances of neighboring media. In some embodiments, there may be one layer or a plurality of layers in the matching layer 110. The matching layer 110 may be set according to actual needs, which is not limited herein. In some embodiments, the matching layer 110 may be provided on an upper surface of the piezoelectric layer 120 by bonding, welding, pinning, or the like. For example, a lower surface of the matching layer may be fixed to the upper surface of the piezoelectric layer by bonding. In some embodiments, the matching layer 110 may be molded and prepared on the basis of the piezoelectric layer 120, such that the molded matching layer 110 may be provided on the upper surface of the piezoelectric layer 120.

In some embodiments, the matching layer 110 may include one or more matching components. For example, as shown in FIG. 1, the matching layer 110 may include a first matching component 110-1, a second matching component 110-2, and/or a third matching component 110-3. In some embodiments, structures, materials, or functions of different matching components may be different, e.g., the sound velocity may be different for different matching components.

The sound velocity refers to a velocity at which a sound signal is transmitted through a medium. In some embodiments, the sound velocity in the matching layer 110 may be the velocity at which the ultrasonic waves are transmitted through the matching layer 110. In a case in which the matching layer 110 includes a plurality of matching components, the sound velocity in the matching layer 110 may refer to sound velocities in different matching components of the matching layer 110. In some embodiments, the sound velocities in the different matching components of the matching layer 110 may be the same or different. For example, as illustrated in FIG. 1, a sound velocity of the first matching component 110-1 in the matching layer 110 and a sound velocity in the third matching component 110-3 may be the same. A sound velocity of the second matching component 110-2 in the matching layer 110 may be different from the sound velocity in the first matching component 110-1 and the sound velocity in the third matching component 110-3. In some embodiments, the different matching components may have fillers with different sound velocities, so that the different matching components have different sound velocities.

Filler(s) of the matching layer 110 may be materials with acoustic properties. In some embodiments, the matching layer 110 may include at least a first filler and a second filler. The first filler may have a first sound velocity. The second filler may have a second sound velocity. The first sound velocity corresponding to the first filler may be different from the second sound velocity corresponding to the second filler. In some embodiments, the first filler and the second filler may be respectively filled at different locations in a same plane of the ultrasonic transducer 100 to achieve a gradient distribution of the sound velocity in the matching layer 110 in at least one direction.

In some embodiments, the first filler and the second filler may be used to form a plurality of matching components, respectively, so that the plurality of matching components may have different sound velocities. Exemplarily, as shown in FIG. 1, the first matching component 110-1 and the third matching component 110-3 may be filled with the first filler, and the second matching component 110-2 may be filled with the second filler, such that the first matching component 110-1 and the third matching component 110-3 may have the first sound velocity and the second matching component 110-2 may have the second sound velocity. In some embodiments, the first filler and the second filler may be filled to a same plane of the ultrasonic transducer 100, which may cause a plurality of matching components to be provided on the same plane of the ultrasonic transducer 100 to form the matching layer 110 of the ultrasonic transducer 100. In some embodiments, the first filler and the second filler may be filled in one or more planes of the ultrasonic transducer 100, to prepare one or more matching layers 110.

It should be noted that the filler (or the component) may be a mixture of one or more materials. The filler and the component may be different terms for the same substance and may refer to the same substance. In some embodiments, the matching component may include only the first filler or the second filler or may include other kinds of materials, such as curing-type materials. Specific descriptions regarding implementing the first filler and the second filler may be found elsewhere in the present disclosure.

In some embodiments, the at least one direction may include a length direction and/or a thickness direction of the piezoelectric array elements of the piezoelectric layer. The length direction of the piezoelectric array element refers to a direction that is parallel to a first edge of a projection of a horizontal plane of the piezoelectric array element. The projection of the horizontal plane of the piezoelectric array element may include a first edge and a second edge, and the first edge is longer than the second side. The direction of the thickness of the piezoelectric array element may refer to a direction perpendicular to the horizontal plane where the piezoelectric array element is located. Exemplarily, the at least one direction may include a Y-axis direction and/or a Z-axis direction in three-dimensional coordinates XYZ. The three-dimensional coordinates may have an intersection of the first edge and the second edge of the piezoelectric array element as an origin, the width direction of the piezoelectric array element as the X-axis, the length direction of the piezoelectric array element as the Y-axis, and the thickness direction of the piezoelectric array element as the Z-axis of the coordinate system. Specific descriptions regarding at least one direction may be found in related descriptions in the three-dimensional coordinates XYZ of FIG. 3 or 11 illustrated below, and may not be repeated here. Specific descriptions regarding the implementation of the piezoelectric array element may be found in related descriptions of the piezoelectric layer hereinafter, and may not be repeated here.

The gradient distribution refers to a distribution of phases or gradients of the sound velocity in one or more directions, such as a monotonically decreasing or increasing, normal distribution, etc. For example, in some embodiments, the gradient distribution of the sound velocity of the matching layer 110 may include that the sound velocity monotonically decreasing or increasing from a first side to a second side of the matching layer 110, decreasing or increasing from a center to a periphery of the matching layer 110, etc. The first side and the second side of the matching layer 110 may be different edges of the matching layer 110, and the first side and the second side may be two edges corresponding to each other. In some embodiments, a direction of the first side to the second side may include the width direction of the matching layer 110 (the Y direction as shown in FIG. 1). In some embodiments, the gradient distribution of the sound velocity of the matching layer 110 may be associated with the thickness distribution of the piezoelectric layer. Detailed descriptions may be found in related descriptions of the piezoelectric layer, and may not be repeated herein.

In some embodiments, when the matching layer 110 has a plurality of layers, different matching layers 110 may have the same gradient distribution of the sound velocity or may have different gradient distributions of the sound velocity. Further, at least one matching layer 110 of the plurality of matching layers 110 may not be in the gradient distribution of the sound velocity.

It should be noted that since the frequency of the ultrasonic wave is negatively correlated with the thickness of the piezoelectric layer, the thickness distribution of the piezoelectric layer is unequal, which results in the ultrasonic waves generated from different regions of the piezoelectric layer having different frequencies. Since the sound velocity of the matching layer 110 is related to the wavelength and frequency of the ultrasonic wave, the equation is as follows: λ=c/f, where λ denotes the wavelength of the ultrasonic wave, c denotes the sound velocity of the matching layer 110, f denotes the frequency of the ultrasonic wave. If the distribution of sound velocity of the matching layer 110 is uniform, wavelengths of the ultrasonic waves corresponding to different frequencies of ultrasonic waves propagating in the matching layer 110 may be different. Besides, a ratio of the thickness of the matching layer 110 to the wavelength of the ultrasonic wave may affect the transmittance. For example, if the thickness of the matching layer 110 is an odd multiple of the quarter wavelength of the ultrasonic wave in the matching layer 110, a desired transmittance may be up to 100%. If the wavelengths of the ultrasonic waves in the matching layer 110 are unequal, the ratio of the thickness of each point of the matching layer 110 to the wavelengths of the ultrasonic waves may be changed, and the acoustic matching may be poor, which may result in a lower transmittance.

In some embodiments of the present disclosure, to make the wavelength of the ultrasonic wave the same in the matching layer, the sound velocity in the matching layer may be set to be in the gradient distribution to realize acoustic matching between the piezoelectric layers of different structures and the human body tissues. Therefore, the transmittance of the ultrasonic waves in the matching layer may be improved, and the bandwidth of the ultrasonic transducer may be increased.

Besides, for ultrasonic waves of different frequencies, the matching layer 110 may be made to have a gradient distribution of the sound velocity by filling fillers having different sound velocities at different locations, so that the ultrasonic waves of different frequencies in the matching layer 110 may be controlled to have the same wavelength. Thus, the transmittance of the matching layer 110 to the ultrasonic waves may be increased and the bandwidth of the ultrasonic transducer 100 may be increased.

It should be noted that negative correlation, inverse correlation, and reverse correlation are different designations of the same relationship, and may refer to the same kind of relationship. In some embodiments, the negative correlation, the inverse relationship, and the reverse relationship refer to relationships between two substances in which the trend of change of the two things is related but the direction of the change is opposite, e.g., one thing increases, and another thing decreases.

In some embodiments, the first filler and the second filler in the matching layer 110 may have the same acoustic impedance and/or the same thickness.

The acoustic impedance may be used to reflect the resistance that needs to be overcome for sound to pass through the medium. In some embodiments, the acoustic impedance of the filler may reflect the resistance that ultrasonic wave needs to overcome when passing through the matching layer 110. For example, the greater the acoustic impedance of the filler, the greater the resistance that the ultrasonic wave needs to overcome when passing through the matching layer 110. Conversely, the smaller the acoustic impedance of the filler, the smaller the resistance that the ultrasonic wave needs to overcome when passing through the matching layer 110.

It should be noted that the difference in acoustic impedance between two objects may reflect whether there is an acoustic matching between the two objects. If the difference in acoustic impedance between two objects exceeds an acoustic impedance threshold, there may be an acoustic mismatch, and the energy of the ultrasonic wave reflected at the boundary of the two media may be greater. On the contrary, if the difference in acoustic impedance between two objects does not exceed the acoustic impedance threshold, there may be an acoustic match, and the energy of the ultrasonic wave reflected at the boundary of the two media may be smaller.

In some embodiments of the present disclosure, by designing the first filler and the second filler to have the same acoustic impedance, it is possible to enable acoustic matching between different fillers. Different matching components in the matching layer 110 (e.g., the first matching component 110-1, the second matching component 110-2, and the third matching component 110-3) may be acoustically matched to reduce the energy consumed by the ultrasonic wave when passing through the matching layer 110.

Thus, the transmission of the ultrasonic wave by the matching layer 110 may be improved.

In some embodiments, when the matching layers 110 have a plurality of layers, the different matching layers 110 may have the same acoustic impedance or may have different acoustic impedances. Exemplarily, the first matching layer may have an acoustic impedance within a range of 8 MRayl-8.5 Mrayl, the second matching layer may have an acoustic impedance within a range of 4 MRayl-6 MRayl, the third matching layer may have an acoustic impedance within a range of 2 MRayl-3 MRayl. Further, in some embodiments, some of the matching layers 110 of the plurality of matching layers 110 may have the same acoustic impedance, and other parts of the matching layers 110 may have different acoustic impedances.

The acoustic impedance of the filler may be affected by the density and the sound velocity. In some embodiments, where different fillers of the matching layer 110 have different sound velocities, the density of the fillers may be adjusted to ensure that the different fillers have the same acoustic impedance. Exemplarily, if the sound velocity of the first filler is greater than the sound velocity of the second filler, the density of the first filler may be decreased or the density of the second filler may be increased so that the first filler and the second filler may have the same acoustic impedance.

In some embodiments, the different fillers of the matching layer 110 may have the same thickness so that the thickness at each point of the matching layer 110 may be equal. For example, as shown in FIG. 1, the matching layer 110 may have an equal distance (i.e., thickness) between two points in the Z-axis direction. Exemplarily, the thickness of the matching layer may be 100 um, 150 um, or 200 um, etc.

In some embodiments of the present disclosure, by setting the thicknesses of the matching layer 110 equal between the different fillers of the matching layer 110, there is no need for special processing of the thickness of the matching layer 110 according to the structure of the piezoelectric layer 120. Therefore, the difficulty of the processing of the ultrasonic transducer 100 may be reduced.

Since the gradient distribution of the sound velocity of the matching layer 110 is associated with the thickness distribution of the piezoelectric layer 120, in some embodiments, a range of the sound velocity of the matching layer 110 may be designed based on the structure of the piezoelectric layer 120 (e.g., the range of thicknesses of different regions of the piezoelectric layer 120). In some embodiments, the first sound velocity of the first filler or the second sound velocity of the second filler may be within a range of 1400 m/s-3500 m/s. Specific descriptions regarding the thickness distribution of the piezoelectric layer 120 may be found in the description related to the piezoelectric layer 120 and may not be repeated herein.

It should be noted that the above first sound velocity and second sound velocity are also used only as examples, and do not limit the order of magnitude of the sound velocity in the matching layer 110, which only indicates that the different fillers in the matching layer 110 have different sound velocity, and may also have a third sound velocity, a fourth sound velocity, etc., which is not limited in the present embodiments.

Fillers are inert substances used to adjust the physical and/or chemical properties of a device, such as changing the sound velocity of a device by adjusting properties such as elastic modulus, density, etc. In some embodiments, the filler (e.g., the first filler and the second filler) may include an inorganic filler (which may also be referred to as an inorganic material) and/or an organic filler (which may also be referred to as an organic material). The sound velocity of the matching layer 110 may be adjusted by changing the type, percentage, size, and/or structure of the material in the filler. The inorganic filler and the organic filler may have different impacts on the sound velocity, and the sound velocity in the matching layer may be adjusted by changing a ratio of the inorganic filler and the organic filler. Exemplarily, the organic filler may include organics such as epoxy resin, silicone rubber, fluorine rubber, nitrile rubber, styrene-butadiene rubber, male-butadiene rubber, ethylene propylene diene monomer (EPDM) rubber, thermoplastic SBS elastomers, a plastic expanding microsphere, etc. The inorganic filler may include non-metallic oxides (e.g., glass, ceramics), metals (e.g., tungsten, copper, iron, etc.), graphite, and/or metal oxides (e.g., tungsten trioxide, aluminum trioxide, iron oxide, etc.) and other inorganic materials. The present disclosure does not limit the specific implementation manner of the filler.

In some embodiments, volume ratios or mass ratios of the plurality of fillers (e.g., the first filler and the second filler, etc.) may be in gradient distribution in the matching layer so that the sound velocity in the matching layer may be in a gradient distribution. The volume ratio of a filler may refer to a ratio of a volume of the filler to a volume of a base material (e.g., epoxy resin). The mass ratio of a filler may refer to a ratio of a mass of the filler to a mass of a base material (e.g., epoxy resin). For example, the first filler may be filled in the first side of the matching layer and a second filler may be filled in the second side of the matching layer, such that the sound velocity corresponding to the first side of the matching layer is different from the sound velocity corresponding to the second side of the matching layer, thereby realizing a gradient distribution that is monotonically increasing or decreasing. For example, as shown in FIG. 1, the first filler may adjust the first matching component 110-1 and the third matching component 110-3 of the matching layer, and the second filler may adjust the second matching component 110-2 of the matching layer. The sound velocity of the first matching component 110-1 of the matching layer is the sound velocity c1, the sound velocity of the third matching component 110-3 of the matching layer is the sound velocity c3, and the sound velocity c1 is the same as the sound velocity c3. The sound velocity of the second matching component 110-2 of the matching layer is the sound velocity c2, and the sound velocity c1 is different from the sound velocity c2.

In some embodiments, the different fillers may include different kinds of materials and/or different structures so that the different fillers may have different properties (e.g., the sound velocity, the density, etc.). In some embodiments, the first filler and the second filler may have at least one different kind of substance. Alternatively, the first filler and the second filler may have the same kind of substance, and at least one of the same kinds of substance may be in a different proportion in the first filler and the second filler, be of different sizes (e.g., different diameters), and/or different structures.

In some embodiments, introducing one or more different kinds of substances into the first filler and/or the second filler may change the sound velocity of the first filler and/or the second filler. In some embodiments, adjusting the percentage, size, or structure of one or more of the same kinds of substances may also change the sound velocity of the first filler and/or the second filler. Further, in some embodiments, adjusting the percentage, size, or structure of different kinds of substances in the first filler and/or the second filler may also adjust the sound velocity of the first filler and/or the second filler.

Merely by way of example, the first filler and the second filler may both include an epoxy resin. To make the first filler and the second filler have different sound velocities, a liquid resin (e.g., a rubbery liquid resin) may be designed to be introduced into the first filler to reduce the sound velocity of the first filler. A metal oxide filler may also be designed to be introduced into the second filler to increase the sound velocity of the second filler. Meanwhile, the sound velocity of the first filler may be further adjusted by adjusting the percentage of the liquid resin in the first filler. Alternatively, the sound velocity of the second filler may be further adjusted by adjusting the percentage of the metal oxide filler in the second filler, the size, and structure of the metal oxide filler.

In the embodiment, by designing the type of substance in the filler, the percentage, the size, the structure, etc., different fillers may have different sound velocities, so as to realize the gradient distribution of the sound velocity of the matching layer 110, and to increase the transmittance of the matching layer 110 to the ultrasonic waves of different frequencies.

The density of the filler may also be affected due to the type and percentage of substances in the filler. In some embodiments, the density of the first filler and/or the second filler may be adjusted due to the type, the percentage of the substances in the first filler and/or the second filler so that the first filler and the second filler may have the same acoustic impedance.

Exemplarily, when reducing the sound velocity of the first filler, a substance (e.g., a metal, etc.) that has a higher density and less effect on the enhancement of the sound velocity may be introduced into the first filler to increase the density of the first filler so that the first filler may have the same acoustic impedance as the second filler. When increasing the sound velocity of the second filler, a substance (e.g., an inorganic non-metallic compound with a hollow structure, etc.) with a lower density and less effect on the reduction of the sound velocity may be introduced into the second filler to reduce the density of the second filler so that the first filler may have the same acoustic impedance as the second filler.

In some embodiments, the first filler and the second filler may be set in the ultrasonic transducer 100 by a process of cast molding, potting molding, injection molding, etc., to form the matching layer 110. In some optional embodiments, the matching layer 110 may be arranged on the piezoelectric layer 120 by bonding, welding, stapling, etc., after the matching layer 110 is obtained by utilizing the above-described preparation process. In some optional embodiments, the matching layer 110 may be prepared on the upper surface of the piezoelectric layer 120 using the above-described preparation process, so that the matching layer 110 may be provided with the piezoelectric layer 120 by adhering after molding. In some embodiments, different fillers may be selected for the matching layer 110 based on different structures of the piezoelectric layer 120 (e.g., with a gradient distribution of thicknesses, etc.) to make the matching layer 110 have a gradient distribution of the sound velocity. Therefore, the matching layer 110 may have a better transmittance for ultrasonic waves with different frequencies, and acoustic matching between the piezoelectric layer 120 with different structures and the object to be measured may be realized.

It should be noted that the first filler and the second filler described above are only examples indicating that there are different fillers in the matching layer 110 and do not limit the order of magnitude of the filler in the matching layer 110. The matching layer 110 may have more other fillers such as a third filler, fourth filler, etc., which is not specifically limited by this embodiment. In some embodiments, other fillers of the matching layer 110 may also have the same properties and functions as the first filler and the second filler.

In some embodiments, different properties of different kinds of substances may be utilized to adjust the sound velocity and/or the density of the first filler and the second filler. In some embodiments, the first filler and/or the second filler may include an epoxy resin. The material of the first filler and/or the second filler may further include a material with a sound velocity in a first sound velocity range, a material with a sound velocity in a second sound velocity range, and at least one of a material with a density in a first density range and a material with a density in a second density range. The first sound velocity range may be less than the second sound velocity range, and the first density range may be greater than the second density range.

The epoxy resin is a thermosetting resin that has good transmission efficiency of the ultrasonic waves. In some embodiments, the epoxy resin may be a substrate for the first filler and/or the second filler to provide an initial sound velocity for the first filler and the second filler. Exemplarily, the epoxy resin has a sound velocity of 2730 m/s and a density of 1.15 g/cm³.

Further, in some embodiments, the first sound velocity range may be less than the sound velocity of the epoxy resin, and the second sound velocity range may be greater than the sound velocity of the epoxy resin. In some embodiments, the first density range may be greater than the density of the epoxy resin, and the second density range may be less than the density of the epoxy resin. That is, adding a material with a sound velocity in the first sound velocity range to the epoxy resin may reduce the sound velocity of the filler, adding a material with a sound velocity in the second sound velocity range may increase the sound speed of the filler, adding a material with a density in the first density range may enhance the density of the filler, and adding a material with a density in the second density range may reduce the density of the filler. Exemplarily, in some embodiments, the first sound velocity range may be from 800 m/s to 2000 m/s, the second sound velocity range may be from 2800 m/s to 11000 m/s, the first density range may be from 3 g/cm³ to 20 g/cm³, and the second density range may be from 0.1 g/cm³ to 0.8 g/cm³.

In some embodiments, the first filler and the second filler may be made of the same kind of material with a sound velocity in the first sound velocity range, the same kind of material with a sound velocity in the second sound velocity range, the same kind of material with a density in the first density range, the same kind of material with a sound density in the second density range, or different kinds of materials. By changing the percentage and content of the materials in different fillers, the first filler and the second filler may have different sound velocities. Further, in some embodiments, the first filler may have the same acoustic impedance as the second filler by changing the percentage and the content of the material with the density in the first density range and the material with the density in the second density range in the different fillers.

It should be noted that the material with the density in the first density range and the material with the density in the second density range may also have different sound velocities, except that the material in the second velocity range has a relatively lower effect on the sound velocity compared to the material with the velocity in the first velocity range. In some embodiments, the first filler and the second filler may include only the material with the density in the first density range and/or the material with the density in the second density range. The percentage and the content of the material with the density in the first density range and/or the material with the density in the second density range may be changed such that the first filler and the second filler may have different sound velocities.

In some embodiments, the material with the sound velocity in the first sound velocity range may include rubber, the material with the sound velocity in the second sound velocity range may include a metal oxide and/or an inorganic non-metallic compound of a solid structure. The material with the density in the first density range may include a metal, the material with the density in the second density range may include an inorganic non-metallic compound of a hollow structure and/or a plastic expanding microsphere. The density of the rubber is similar to the density of the epoxy resin, and the density of the rubber has a low sound velocity, in some embodiments, the rubber may have a relatively lower effect on the density and a greater effect on the sound velocity and may be used to reduce the sound velocity of the filler. For example, the sound velocity of the ultrasonic wave in the rubber is about 950 m/s to 2400 m/s. Adding the rubber to the epoxy resin (i.e., a base of the filler) may reduce the sound velocity of the filler.

In some embodiments, the metal may have a relatively lower effect on the sound velocity and a relatively greater effect on the density and may be used to increase the density of the filler. In some embodiments, if the density of the added metal is relatively low, a larger volume fraction of the metal needs to be added, such that the addition of a larger volume fraction of the metal with a lower density, as compared to other metals with a higher density may further increase the sound velocity of the filler. In some embodiments, the metal oxide may have a relatively lower effect on the density and a relatively greater effect on the sound velocity and may be used to increase the sound velocity of the filler.

In some embodiments, the inorganic non-metallic compound of the hollow structure (e.g., hollow ceramic microspheres, hollow glass beads) may have a relatively lower effect on the sound velocity and a relatively greater effect on the density and may be used to reduce the density of the filler. In some embodiments, the inorganic non-metallic compound of the solid structure (e.g., solid ceramic beads, solid boron carbide beads, etc.) may have a relatively lower effect on the density and a relatively greater effect on the sound velocity and may be used to increase the sound velocity of the filler.

The plastic expanding microsphere may have a relatively lower effect on the sound velocity and a relatively greater effect on the density. The density of the plastic expanding microsphere may be less than the density of the epoxy resin. In some embodiments, the plastic expanding microsphere may be used to reduce the density of the filler. For example, if the density of the plastic expanding microsphere may be within a range of approximately 0.1 g/cm³-0.13 g/cm³, the addition of the plastic expanding microsphere to the epoxy resin (i.e., the substrate of the filler) may reduce the density of the filler.

In some embodiments, the sound velocity of the filler may also be adjusted by adjusting a particle size of the plastic expanding microsphere. With the material increases, the sound velocity increases as the particle size of the plastic expanding microsphere increases. In some embodiments, the metal, the metal oxide, or the inorganic non-metallic compound may be added to the epoxy resin in the form of a powder or a microsphere (also referred to as a microbead), etc.

In some embodiments, the material of the first filler may include the epoxy resin, the rubber, and the metal. The material of the second filler may include the epoxy resin, metal oxide, and the inorganic non-metallic compound of the hollow structure. The first filler may have a first sound velocity less than the second sound velocity of the second filler. The first filler and the second filler may have the same acoustic impedance. In other words, the addition of the rubber with a lower sound velocity and a metal with a higher density to the epoxy resin makes it possible to make the first filler have a low sound velocity and a high density. Adding a metal oxide with a higher sound velocity and an inorganic non-metallic compound of a hollow structure with a lower density to the epoxy resin may cause the second filler to have a high sound velocity and a low density, so that the first sound velocity of the first filler may be less than the second sound velocity of the second filler. The acoustic impedance of the filler is affected by the density and the sound velocity, further, in some embodiments, the first filler with a low sound velocity and high density, and the second filler with a high sound velocity and low density may have the same acoustic impedance.

Several exemplary substances of the above materials are provided below, respectively, to describe in detail specific implementations of the filler.

In some embodiments, the epoxy resin may include at least one of an epoxy resin of a bisphenol A type or an epoxy resin of a bisphenol F type. The rubber may include at least one of thermoplastic SBS elastomer, nitrile rubber, a butyl rubber, styrene-butadiene rubber, male-butadiene rubber, ethylene propylene diene monomer (EPDM) rubber, silicone rubber, or fluorine rubber. The metal may include at least one of tungsten, copper, iron, or lead. The metal oxide may include at least one of a tungsten trioxide, an iron oxide, an aluminum trioxide, a zinc oxide, or a magnesium oxide. The inorganic non-metallic compound may include at least one of glass, ceramic, and or boron carbide.

In some embodiments, the content of the epoxy resin may be 100 g. The content of the material with the sound velocity in the first sound velocity range may be less than or equal to 60 g, or the content of the material with the sound velocity in the second sound velocity range may be less than or equal to 130 g, or the content of the material with the sound velocity in the first density range may be less than or equal to 500 g, or the content of the material with the sound velocity in the second density range may be less than or equal to 20 g. Furthermore, in some embodiments, the content of the rubber may be less than or equal to 60 g, the content of the metal may be less than or equal to 500 g, or the content of the metal oxide may be less than or equal to 130 g, or the content of the inorganic non-metallic compound of the solid structure may be less than or equal to 130 g, or the content of the inorganic non-metallic compound of the hollow structure may be less than or equal to 20 g, or the content of the plastic expanding microsphere may be less than or equal to 20 g.

It is to be noted that the substances provided above are only exemplary, and the filler may also include other substances with similar functions and properties. The specific implementation of the filler may be referred to elsewhere in the present disclosure and may not be repeated herein.

In some embodiments, the filler may be provided in the matching layer 110 by a process such as injection molding, etc. In some embodiments, different fillers may be selected for the matching layer 110 based on different structures of the piezoelectric layer 120 (e.g., the gradient distribution of the thickness, etc.) to achieve acoustic matching between the piezoelectric layer 120 of different structures and the object to be measured.

In some optional embodiments, the thickness at various points of the matching layer 110 may be equal. For example, as shown in FIG. 1, the matching layer 110 has an equal distance (i.e., thickness) between two points in a Z-axis direction.

In some embodiments of the present disclosure, by setting the thickness of the matching layer 110 equal at all points, there is no need to perform special processing on the thickness of the matching layer 110 according to the structure of the piezoelectric layer 120, thereby reducing the difficulty of the processing process of the ultrasonic transducer.

In some optional embodiments, the thickness of the matching layer 110 may be unequal. Different thicknesses of the matching layer 110 may be that the thickness of the matching layer 110 includes two or more thicknesses. In some embodiments, the thickness of the matching layer 110 may include a fifth thickness and a sixth thickness, and the fifth thickness may be unequal to the sixth thickness. In some embodiments, the unequal thicknesses of the matching layer 110 may refer to the unequal thicknesses at various points of the matching layer 110, such as the thicknesses of the matching layer 110 may be changed continuously. Exemplarily, the thickness of the matching layer 110 may be at least one of 100 μm, 150 μm, 200 μm, etc. The present disclosure does not limit the specific implementation of the thickness of the matching layer 110. The specific details of the thickness of the matching layer 110 may be referred to in the relevant descriptions in the following FIGS. 3-18, and may not be repeated here.

It should be noted that the fifth thickness and the sixth thickness do not qualify the order of magnitude of the thickness of the matching layer 110, but only indicate that there is a different thickness of the matching layer 110. The thickness of the matching layer 110 may also include other thicknesses such as a seventh thickness, an eighth thickness, etc., which are not specifically limited by the present embodiment.

In some embodiments of the present disclosure, by setting the matching layer 110 with different thicknesses, it is possible to make the thickness of the matching layer 110 cooperate with the sound velocity, to make the wavelength of the ultrasonic wave in the matching layer 110 consistent, and thus to improve the transmittance of the ultrasonic wave to realize an ultrasonic different structural design of the transducer.

The piezoelectric layer 120 may be a layered structure fabricated utilizing a device having a piezoelectric effect. In some embodiments, a piezoelectric wafer may be made from one or more of piezoelectric crystals (e.g., quartz crystals, lithium iodate, etc.), piezoelectric semiconductors (e.g., cadmium sulfide, zinc oxide, etc.), piezoelectric ceramics, piezoelectric composites, piezoelectric polymers, etc., which is not limited in the embodiments of the present disclosure. In some embodiments, the piezoelectric layer 120 may include a piezoelectric device in a shape of a circular sheet, an elongated sheet, a rod, a cylinder, etc.

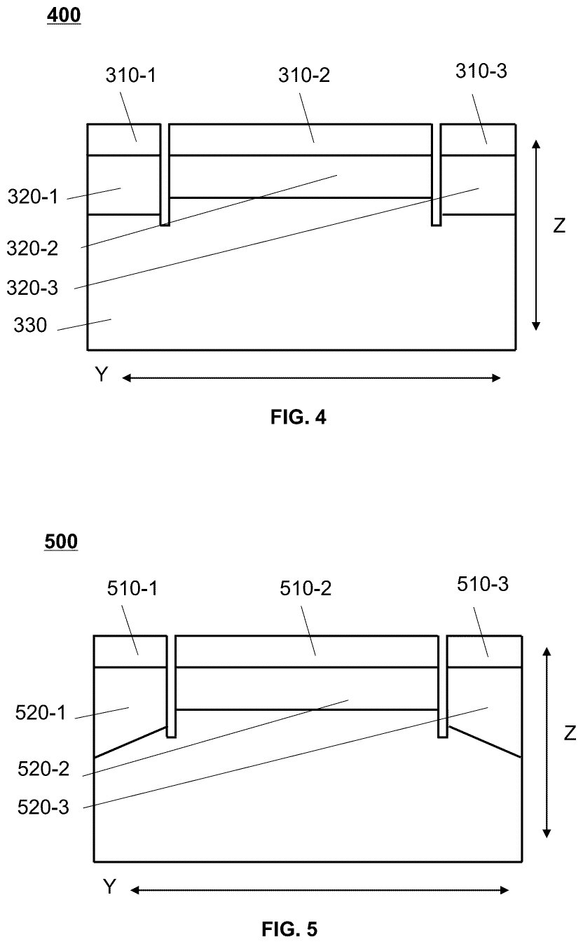

In some embodiments, the piezoelectric layer 120 may include one or more piezoelectric wafers, and the piezoelectric wafer may be a device having a piezoelectric effect. In some embodiments, the piezoelectric wafer may serve as the piezoelectric layer 120, as shown in FIG. 1. In some embodiments, when there are a plurality of piezoelectric wafers, the plurality of piezoelectric wafers may be made into the piezoelectric layer 120 by stacking, such as stacking by vertical stacking, horizontal arrangement, and multiple arrays side by side. The specific implementation of the plurality of piezoelectric wafers may be referred to in the relevant descriptions in the following FIG. 4 and may not be repeated here.

In some embodiments, the piezoelectric layer 120 may include one or more piezoelectric array elements.

The piezoelectric array element may be an element of an array structure of the piezoelectric layer 120, which may be divided based on the smallest operation unit of the piezoelectric layer 120. In some embodiments, the array structure of the piezoelectric layer 120 may include a single array, a double array, a triple array, a quadruple array, a quintet array, a five-array, and other multiple-array side-by-side structures, and the present disclosure does not make any specific limitation as to the number of arrays. The arrays may be a collection of the same columns of piezoelectric array elements stacked along a width direction of the piezoelectric array elements. In some embodiments, the width direction of the piezoelectric array elements may refer to a direction parallel to a second edge of a projection of a horizontal plane of the piezoelectric array elements, and a first edge of the projection of the horizontal plane of the piezoelectric array elements may be longer than the second edge. Exemplarily, the width direction of the piezoelectric array element may be the X-axis direction of the three-dimensional coordinates XYZ. The specific implementation of the width direction may be referred to in the following FIG. 3 and may not be repeated here.

In some embodiments, one piezoelectric array element may include one or more piezoelectric wafers. For example, one piezoelectric array element in the piezoelectric layer 120 may include one piezoelectric wafer, as shown in FIG. 1. Other specific implementations of the piezoelectric array element may be referred to in FIGS. 1-10 below and may not be repeated here.

In some embodiments, the thickness of the piezoelectric layer 120 may be related to the thickness of the piezoelectric wafer. In some embodiments, when the piezoelectric wafers are stacked by way of a horizontal arrangement or multiple arrays side-by-side, the thickness of the piezoelectric layer 120 may be equal to the thickness of the piezoelectric wafers. For example, the piezoelectric layer 120 includes one piezoelectric wafer as shown in FIG. 1, and the thickness of the piezoelectric layer 120 may be equal to the thickness of the piezoelectric wafer.

In some embodiments, the thickness of the piezoelectric layer 120 may vary. The unequal thicknesses of the piezoelectric layer 120 may refer to that the thickness of the piezoelectric layer 120 includes two or more thicknesses. In some embodiments, the thickness of the piezoelectric layer 120 may include a first thickness and a second thickness, and the first thickness may be unequal to the second thickness. For example, as shown in FIG. 1, the thickness of a middle portion of the piezoelectric layer 120 (i.e., the first thickness) may be less than thicknesses of two ends (i.e., the second thickness) of the piezoelectric layer 120. It should be noted that the first thickness and the second thickness do not limit the count of thicknesses of the piezoelectric layer 120, but only indicate that there are different thicknesses of the piezoelectric layer 120. The thickness of the piezoelectric layer 120 may also include other thicknesses, such as a seventh thickness, an eighth thickness, etc., which are not specifically limited by the embodiment.

In some embodiments, the thickness distribution of the piezoelectric layer 120 may be the gradient distribution of thickness. In some embodiments, the thickness distribution of the piezoelectric layer 120 may include a thickness monotonically increasing or monotonically decreasing from a first side of the piezoelectric layer 120 to a second side, circumferentially increasing or circumferentially decreasing from a center of the piezoelectric layer 120, etc. In some embodiments, the thickness monotonically increases or monotonically decreases from the first side to the second side of the piezoelectric layer 120 may include that the thickness monotonically increases or decreases in a stepwise or a straight line manner. In some embodiments, the thickness circumferentially increases or circumferentially decreases from the center of the piezoelectric layer 120 may include that the thickness circumferentially increases or circumferentially decreases in a stepwise, a straight line, a curve, a parabolic curve manner, etc. In some embodiments, the thickness distribution of the above-described piezoelectric layer 120 may be set according to the actual needs, and the present embodiment is not limited herein. The specific implementation of the thickness distribution of the piezoelectric layer 120 may be referred to the relevant contents shown in FIGS. 3-10 below and may not be repeated herein.

In some embodiments, the piezoelectric layer may also be of equal thickness. The specific implementation of the equal thickness of the piezoelectric layer may be referred to in the relevant contents shown in FIGS. 11-18 below and may not be repeated here.

As different thicknesses of the piezoelectric layer 120 may lead to different frequencies of the generated ultrasonic waves, in some embodiments, a gradient distribution of the sound velocity of the matching layer 110 may be associated with a thickness distribution of the piezoelectric layer 120. In some embodiments, the gradient distribution of the sound velocity of the matching layer 110 may be set according to the thickness distribution of the piezoelectric layer 120 so that the wavelength of the ultrasonic wave may be the same in the matching layer. In some embodiments, the sound velocity of the matching layer 110 may include a first sound velocity and a second sound velocity. When the first sound velocity is greater than the second sound velocity and the first thickness of the piezoelectric layer 120 is less than that of the second thickness, the first sound velocity of the matching layer 110 may be associated with the first thickness of the piezoelectric layer, and the second sound velocity of the matching layer 110 may be associated with the second thickness of the piezoelectric layer. In some embodiments, the sound velocity of the matching layer 110 may be inversely proportional to the thickness of the piezoelectric layer 120 at a corresponding location.

For example, as shown in FIG. 1, in the Y-axis direction, the middle portion of the piezoelectric layer 120 may be associated with the second matching component 110-2 of the matching layer, a left portion of the piezoelectric layer may be associated with the first matching component 110-1 of the matching layer, and the right portion of the piezoelectric layer may be associated with the third matching component 110-3 of the matching layer. As shown in FIG. 1, the matching layer 110 is of equal thickness at all points. To make the wavelength of the ultrasonic wave be the same in the matching layer, the distribution of the sound velocity of the matching layer 110 may be negatively correlated to the thickness distribution of the piezoelectric layer. The sound velocity of the second matching component 110-2 of the matching layer may be higher than the sound velocity of the first matching component 110-1 and the sound velocity of the third matching component 110-3 in the matching layer. The sound velocity c2 of the second matching component 110-2 in the matching layer may be associated with the first thickness of the piezoelectric layer, and the sound velocity c1 of the first matching component 110-1 and the sound velocity c3 of the third matching component 110-3 in the matching layer may be associated with the second thickness of the piezoelectric layer. The gradient distribution of the sound velocity in the matching layer circumferentially decreasing from the center may be associated with the thickness distribution of the piezoelectric layer circumferentially increasing from the center.

In some embodiments, the first sound velocity of the first filler in the matching layer 110 may be associated with the first thickness of the piezoelectric layer 120, and the second sound velocity of the second filler in the matching layer 110 may be associated with the second thickness of the piezoelectric layer 120. The first thickness may be greater than the second thickness, and the first sound velocity may be less than the second sound velocity. Descriptions regarding the specific implementation of the sound velocity distribution being associated with the thickness of the piezoelectric layer may be found in related descriptions in FIGS. 3-10 below and may not be repeated here.

In some embodiments of the present disclosure, the gradient distribution of the sound velocity in the matching layer may be set according to the thickness distribution of the piezoelectric layer, so that the wavelength of the ultrasonic wave may be the same in the matching layer. Therefore, the transmittance of the ultrasonic wave of the object to be measured may be improved, and the piezoelectric layer and the object to be measured may be acoustically matched.

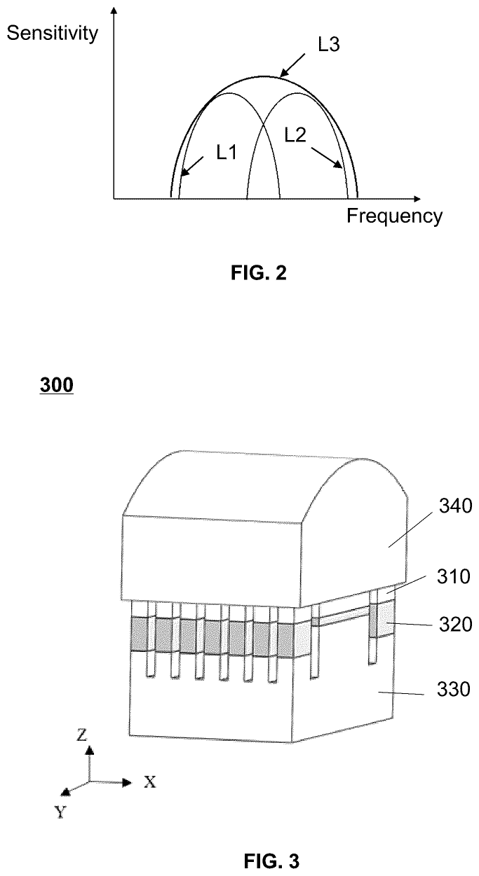

FIG. 2 is a graph illustrating exemplary frequency characteristic curves of an ultrasonic transducer according to some embodiments of the present disclosure.

In some embodiments, as shown in FIG. 2, the L2 curve is a frequency curve of the ultrasonic wave corresponding to a middle portion of the piezoelectric layer 120 as shown in FIG. 1. The L1 curve is a frequency curve of the ultrasonic wave corresponding to a left portion and a right portion of the piezoelectric layer 120 as shown in FIG. 1. The L3 curve is a frequency curve of the ultrasonic wave of the ultrasonic transducer 100 shown in FIG. 1. The frequency curve may represent a correspondence between the frequency of the ultrasonic wave and the sensitivity. The sensitivity may represent a degree of the object to be measured in response to the ultrasonic wave. For example, the sensitivity may represent a ratio of an amplitude value of the ultrasonic wave reflected by the object to be measured to an amplitude value of the ultrasonic wave emitted by the ultrasonic transducer. In some embodiments, a higher sensitivity of the frequency curve may indicate a greater response of the object to be measured to the ultrasonic wave. In some embodiments, a resonance frequency of the L2 curve may be higher than a resonance frequency of the L1 curve. The resonance frequency may be a frequency corresponding to the highest sensitivity of the frequency curve. In other words, the frequency of the ultrasonic wave is related to the thickness of the piezoelectric layer. In some embodiments, the bandwidth of the L3 curve may be greater than the bandwidth of the L1 curve and the L2 curve. That is, the ultrasonic transducer provided by embodiments of the present disclosure may have a greater bandwidth as compared to existing ultrasonic transducer having a single resonant frequency.

It should be noted that, based on the acoustic impedance matching, the acoustic impedance of the first matching component 110-1, the acoustic impedance of the second matching component 110-2, and the acoustic impedance of the third matching component 110-3 of the matching layer may be designed to be the same. As described above, if the thickness of the matching layer is an odd multiple of the quarter wavelength of the ultrasonic wave in the matching layer, the transmittance may be higher, and the sound velocity of the second matching component 110-2 of the matching layer may be higher than the sound velocity of the first matching component 110-1 and the third matching component 110-3 of the matching layer. Then, the structure of the ultrasonic transducer having the same thickness as the matching layer as shown in FIG. 1 may be obtained, so that the frequency curve of the ultrasonic wave is as shown in the L3 curve in FIG. 2, thus ensuring a certain ultrasonic wave transmittance.

In some embodiments of the present disclosure, acoustic matching between piezoelectric layers with different structures and human tissues may be achieved by the gradient distribution of the sound velocity in the matching layer, which increases the transmittance of the ultrasonic wave in the matching layer and increases the bandwidth of the ultrasonic transducer.

In some embodiments, the piezoelectric layer 120 may be of a different structure and/or composition. For example, the piezoelectric layer 120 may include a plurality of shapes. The piezoelectric layer 120 may be set up according to actual needs, which is not limited in the present embodiment.

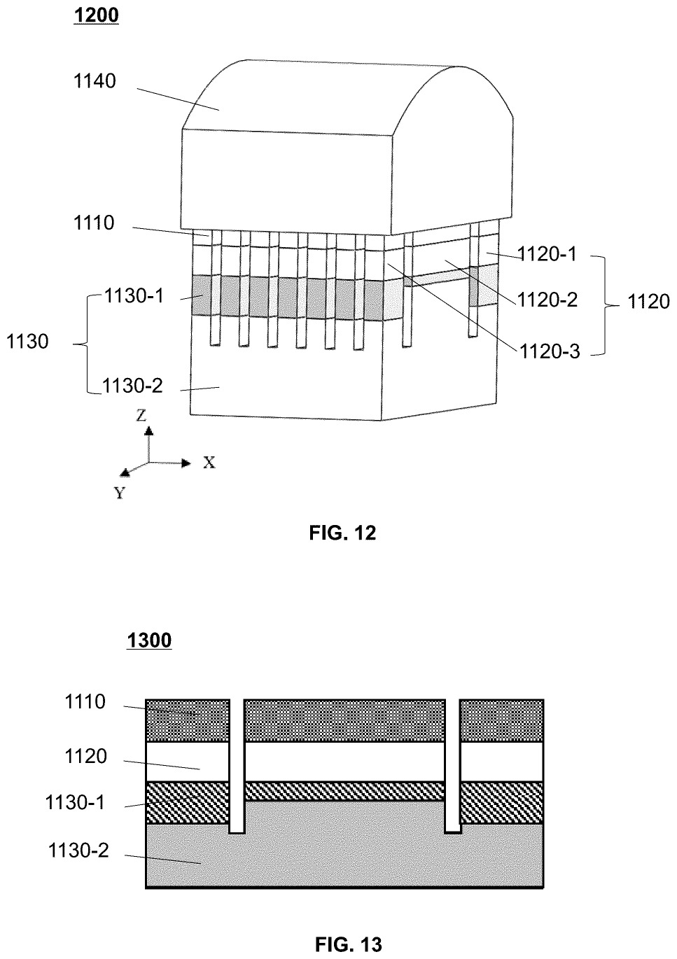

In some embodiments, the thickness of the piezoelectric layer 120 at various points may be the same, and the ultrasonic transducer 100 may transmit and receive ultrasonic waves of different frequencies through the thickness distribution of other structures, such as a backing with a high impedance in a backing layer 130 described below. Correspondingly, in some embodiments, the distribution of the sound velocity in the matching layer 110 may be associated with the distribution of the thickness of the backing with the high impedance in the backing layer 130. Descriptions regarding the specific implementation may be found in FIGS. 11-12 and may not be repeated here.

In some embodiments, the ultrasonic transducer 100 may further include the backing layer 130, as shown in FIG. 1. The backing layer 130 may be provided on a side of the piezoelectric layer 120 away from the matching layer 110.

The backing layer 130 may be of a layered structure that absorbs the ultrasonic wave generated by the piezoelectric layer in a direction opposite to the direction of the object to be measured. In some embodiments, the backing layer 130 may include a plurality of materials, such as one or a combination of materials such as metals, metal oxides, organic materials, etc.

In some embodiments, when the piezoelectric wafer of the piezoelectric layer 120 is stimulated to emit ultrasonic waves, ultrasonic waves propagated in a part of directions may enter the backing layer 130 and may be strongly reflected at the backing layer 130, and the strongly reflected ultrasonic wave may propagate forward through the piezoelectric layer 120. Descriptions regarding implementing the backing layer 130 may be found in FIGS. 11-12 and related descriptions thereof and may not be repeated here.

The following are examples from FIGS. 3-10 to illustrate in detail the specific correspondence between the gradient distribution of the sound velocity in the matching layer and the thickness distribution of the piezoelectric layer when the matching layer is of a uniform thickness.

FIG. 3 is a schematic diagram illustrating an exemplary three-dimensional structure of a first type of ultrasonic transducer according to some embodiments of the present disclosure.

In some embodiments, the ultrasonic transducer 300 includes a matching layer 310, a piezoelectric layer 320, a backing layer 330, and an acoustic lens 340, as shown in FIG. 3. In the Z-axis direction, an acoustic lens 340, the matching layer 310, the piezoelectric layer 320, and the backing layer 330 may be stacked from top to bottom.

In some embodiments, the ultrasonic transducer may further include an acoustic lens, and the acoustic lens may be provided on a side of the matching layer away from the piezoelectric layer. The acoustic lens refers to an acoustic element that converges or diverges sound waves. In some embodiments, the acoustic lens may change a transmission direction of the ultrasonic wave, i.e., refraction occurs, causing the ultrasonic wave to converge or diverge. In some embodiments, the acoustic lens may make the ultrasonic waves emitted by the matching layer converge. The present disclosure does not specifically limit the acoustic lens.

FIG. 4 is a schematic diagram illustrating an exemplary structure of a first type of ultrasonic transducer according to some embodiments of the present disclosure. The matching component in the ultrasonic transducer 400 illustrated in FIG. 4 is similar to the matching component in the ultrasonic transducer 100 illustrated in FIG. 1 above, which may be implemented by referring to FIG. 1 for the implementation of the matching component.

In some embodiments, the thickness of the piezoelectric layer may increase or decrease from the center of the piezoelectric layer toward a periphery. For example, as shown in FIG. 4, a second piezoelectric array element 320-2 in the center of the piezoelectric layer is a first thickness, and the first piezoelectric array element 320-1 and the third piezoelectric array element 320-3 at the periphery of the piezoelectric layer is a second thickness. The first thickness is less than the second thickness. The sound velocity of a second matching component 310-2 of the matching layer is a first sound velocity, and the sound velocity of a first matching component 310-1 and a third matching component 310-3 of the matching layer is a second sound velocity. The first sound velocity may be greater than the second sound velocity. In a Y-axis direction, the first sound velocity of the second matching component 310-2 of the matching layer may be associated with the first thickness of the piezoelectric layer 320-2. The second sound velocity of the first matching component 310-1 and the third matching component 310-3 of the matching layer may be associated with the second thickness of the first piezoelectric array element 320-1 and the third piezoelectric array element 320-3 of the piezoelectric layer. That is to say, the gradient distribution of the sound velocity in the matching layer decreasing from the center to the periphery may be associated with the thickness distribution of the piezoelectric layer increasing from the center to the periphery.

FIG. 5 is a schematic diagram illustrating an exemplary structure of a first type of piezoelectric layer of an ultrasonic transducer according to some embodiments of the present disclosure.

In some embodiments, as shown in FIG. 5, the center of the piezoelectric layer (e.g., the second piezoelectric array element 520-2) is the first thickness, and the periphery of the piezoelectric layer (e.g., the first piezoelectric array element 520-1 and the third piezoelectric array element 520-3) is the second thickness. The second thickness is incrementally increased from the edge of the second piezoelectric array element 520-2 to the periphery, and the first thickness is less than a minimum value of the second thickness. The sound velocity of the second matching component 510-2 of the matching layer is the first sound velocity, and the sound velocity of the first matching component 510-1 and the third matching component 510-3 of the matching layer is the second sound velocity. The second sound velocity decreases from the edge of the second matching component 510-2 of the matching layer to the periphery of the matching layer, and the first sound velocity is greater than a maximum value of the second sound velocity. In the Y-axis direction, the first sound velocity of the second matching component 510-2 of the matching layer may be associated with the first thickness of the piezoelectric layer 520-2. The second sound velocity of the first matching component 510-1 and the third matching component 510-3 of the matching layer may be associated with the second thickness of the first piezoelectric array element 520-1 and the third piezoelectric array element 520-3 of the piezoelectric layer. That is to say, the gradient distribution of the sound velocity of the matching layer decreasing from the center to the periphery may be associated with the thickness distribution of the piezoelectric layer increasing from the center to the periphery.

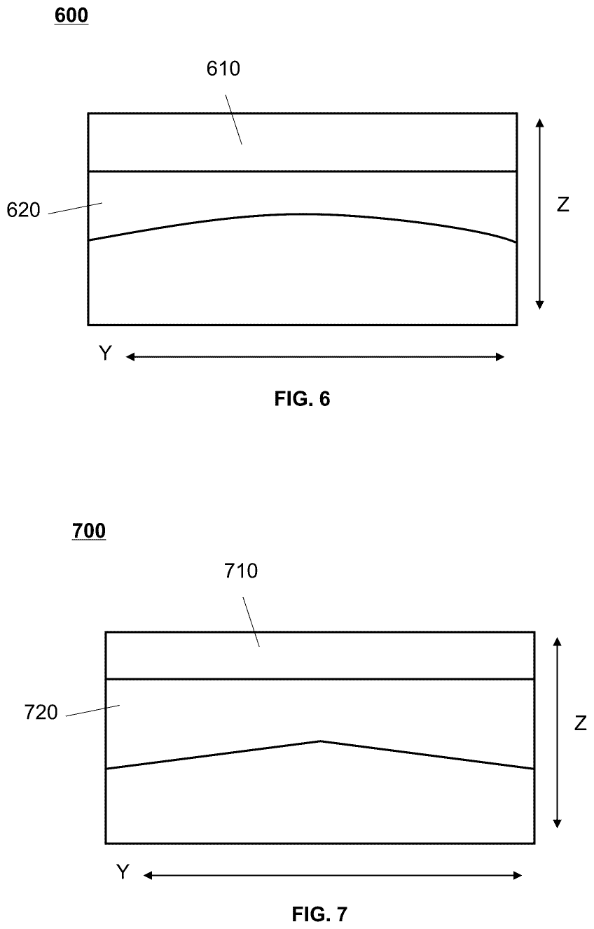

FIG. 6 is a schematic diagram illustrating an exemplary structure of a second type of piezoelectric layer of an ultrasonic transducer according to some embodiments of the present disclosure. FIG. 7 is a schematic diagram illustrating an exemplary structure of a third type of piezoelectric layer of an ultrasonic transducer according to some embodiments of the present disclosure.

In some embodiments, the first thickness of the piezoelectric layer may be a minimum thickness of the piezoelectric layer, and the second thickness of the piezoelectric layer may be a maximum thickness of the piezoelectric layer, as shown in FIG. 6 or 7. The thickness distribution of the piezoelectric layer increases from the center of the piezoelectric layer to the periphery in a curved (e.g., FIG. 6) or straight (e.g., FIG. 7) pattern. In some embodiments, the thickness distribution of the piezoelectric layer described above may be set according to actual needs, which is not limited in the present embodiment. In some embodiments, the sound velocity in the matching layer decreasing from the center to the periphery may be associated with the thickness of the piezoelectric layer increasing from the center to the periphery. As shown in FIG. 6, the gradient distribution of the sound velocity of the matching layer 610 is associated with the thickness distribution of the piezoelectric layer 620. That is, the sound velocity decreasing (e.g., decreasing by curve) from the center to the periphery of the matching layer 610 is associated with the thickness of the piezoelectric layer 620 increasing (e.g., increasing by curve) from the center to the periphery.

In some embodiments, the gradient distribution of the sound velocity of the matching layer 710 may be associated with the thickness distribution of the piezoelectric layer 720, as shown in FIG. 7. That is, the sound velocity decreasing (e.g., linearly decreasing) from the center to the periphery of the matching layer 710 is associated with the thickness of the piezoelectric layer 720 increasing (e.g., linearly increasing) from the center to the periphery.

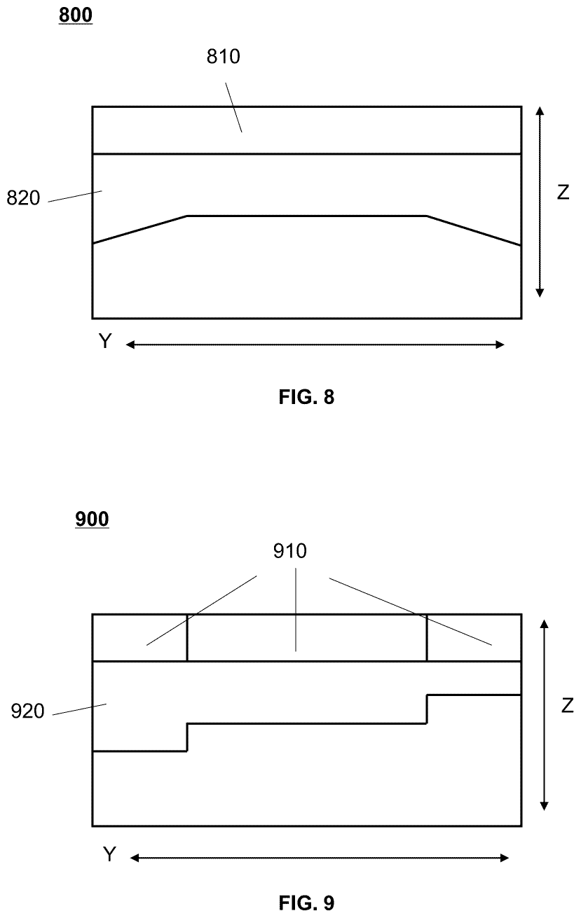

FIG. 8 is a schematic diagram illustrating an exemplary structure of a fourth type of piezoelectric layer of an ultrasonic transducer according to some embodiments of the present disclosure.

In some embodiments, as shown in FIG. 8, a thickness of the center piezoelectric array elements of the piezoelectric layer 820 may have a first thickness. A thickness of the periphery piezoelectric array elements of the piezoelectric layer 820 may change (from the inside to the outside) from the first thickness to the second thickness. The second thickness may be greater than the first thickness. That is, the sound velocity of the matching layer 810 decreasing from the center to the periphery may be associated with the thickness of the piezoelectric layer increasing from the center to the periphery.

FIG. 9 is a schematic diagram illustrating an exemplary structure of a fifth type of piezoelectric layer of an ultrasonic transducer according to some embodiments of the present disclosure.

In some embodiments, the thickness of the piezoelectric layer may decrease monotonically from a first side of the piezoelectric layer to a second side. For example, as shown in FIG. 9, the thickness of the piezoelectric layer 920 decreases monotonically step by step from a left side of the piezoelectric layer (i.e., the first side of the piezoelectric layer) to a right side (i.e., the second side of the piezoelectric layer). In some embodiments, the sound velocity of the matching layer monotonically increasing from the first side to the second side may be associated with the thickness of the piezoelectric layer monotonically decreasing from the first side to the second side. As shown in FIG. 9, the gradient distribution of the sound velocity of the matching layer 910 is associated with the thickness distribution of the piezoelectric layer 920. That is, the sound velocity of the matching layer 910 decreasing monotonically step by step from the first side (the left side) to the second side (the right side) is associated with the thickness of the piezoelectric layer 920 decreasing monotonically step by step from the first side (the left side) to the second side (the right side).

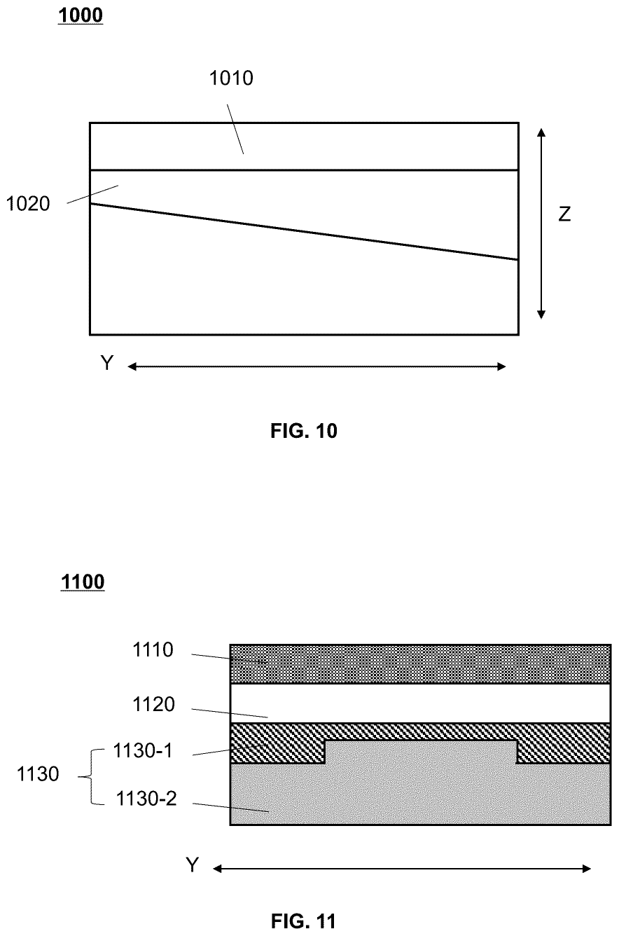

FIG. 10 is a schematic diagram illustrating an exemplary structure of a sixth type of piezoelectric layer of an ultrasonic transducer according to some embodiments of the present disclosure.

In some embodiments, the thickness of the piezoelectric layer may increase monotonically from the first side to the second side of the piezoelectric layer. For example, as shown in FIG. 10, the thickness of the piezoelectric layer 1020 increases monotonically in a straight line from the left side (i.e., the first side of the piezoelectric layer) to the right side (i.e., the second side of the piezoelectric layer) of the piezoelectric layer. In some embodiments, the sound velocity of the matching layer monotonically decreasing from the first side to the second side may be associated with the thickness of the piezoelectric layer monotonically increasing from the first side to the second side. As shown in FIG. 10, the gradient distribution of the sound velocity of the matching layer 1010 is associated with the thickness distribution of the piezoelectric layer 1020. The sound velocity of the matching layer 1010 monotonically decreasing from the first side (the left side) to the second side (the right side) is associated with the thickness of the piezoelectric layer 1020 monotonically increasing from the first side (the left side) to the second side (the right side).

FIG. 11 is a schematic diagram illustrating an exemplary structure of a second type of ultrasonic transducer according to some embodiments of the present disclosure.

In some embodiments, the backing layer 1130 may include a first impedance layer 1130-1 and a second impedance layer 1130-2. The first impedance layer 1130-1 may be connected to the second impedance layer 1130-2. The first impedance layer 1130-1 may be connected to a surface of the piezoelectric layer 1120 away from the matching layer 1110. The first impedance layer may have a higher impedance than the second impedance layer.

In some embodiments, a lower surface of the matching layer 1110 of the ultrasonic transducer may be bonded to an upper surface of the piezoelectric layer 1120. The backing layer 1130 may include a first impedance layer 1130-1 and a second impedance layer 1130-2. The upper surface of the first impedance layer 1130-1 may be bonded to the lower surface of the piezoelectric layer 1120, and a lower surface of the first impedance layer 1130-1 may be bonded to an upper surface of the second impedance layer 1130-2. When the piezoelectric wafer of the piezoelectric layer 1120 is stimulated to emit ultrasonic waves, ultrasonic waves propagated in a part of the directions may enter the first impedance layer 1130-1 to form a back acoustic wave. The back acoustic wave may be strongly reflected at an interface of the first impedance layer 1130-1 and the second impedance layer 1130-2, so that the strongly reflected back acoustic wave may be propagated forward through the piezoelectric layer 1120. In some embodiments, the back acoustic wave may be a portion of the ultrasonic waves that are propagated in an opposite direction of a predetermined propagation direction. For example, the predetermined propagation direction of the ultrasonic waves emitted by the piezoelectric layer 1120 is a direction from the piezoelectric layer 1120 to the matching layer 1110. The propagation direction of the backing acoustic wave corresponding to the ultrasonic waves emitted by the piezoelectric layer 1120 is from the piezoelectric layer 1120 to the backing layer 1130. In some embodiments, the piezoelectric layer 1120 and the first impedance layer 1130-1 may be regarded as an equivalent oscillator, with a resonant frequency of the oscillator being negatively correlated with the thickness of the first impedance layer 1130-1. In some embodiments, the gradient distribution of the sound velocity in the matching layer 1110 may be negatively correlated with a thickness distribution of a thickness sum of the first impedance layer 1130-1 and the piezoelectric layer 1120.

In some embodiments, the backing layer 1130 may include a plurality of materials. For example, the first impedance layer 1130-1 may include a material that has a high impedance and a low acoustic attenuation coefficient, such as a metal, a metal oxide, etc., or a mixture thereof. The second impedance layer 1130-2 may include a material with a low impedance and a high acoustic attenuation coefficient, such as an organic material, etc.

In some embodiments, the impedance of the first impedance layer 1130-1 may be 10 to 40 times the impedance of the second impedance layer 1130-2. It should be noted that impedance multiples herein are only exemplary and are not limited in the present disclosure.

In some embodiments, the thickness of the piezoelectric layer may be equal at various points, and the thickness of the first impedance layer may be unequal. The unequal thickness of the first impedance layer refers to a thickness of two or more thicknesses of the first impedance layer. In some embodiments, the thickness of the first impedance layer may include a third thickness and a fourth thickness, and the third thickness may be unequal to the fourth thickness. For example, the thickness of the piezoelectric layer 1120 may be equal at various points, as shown in FIG. 12. The thickness of the first impedance layer 1130-1 may include a third thickness (a middle portion of the first impedance layer 1130-1) and a fourth thickness (a left portion and a right portion of the first impedance layer 1130-1), and the third thickness may be less than the fourth thickness. It should be noted that the third thickness and the fourth thickness do not qualify the count of thicknesses of the first impedance layer, but only indicate that there are different thicknesses of the first impedance layer. The thickness of the first impedance layer may also include other thicknesses, such as a ninth thickness, a tenth thickness, etc., which are not specifically limited in the present embodiment.

In some embodiments of the present disclosure, a backing structure with a low impedance and a structure with a high impedance may cooperate to realize the transmitting and receiving of ultrasonic waves of different frequencies by changing the thickness of the backing structure with the high impedance.

In some embodiments, the thickness of the first impedance layer 1130-1 may be in a gradient distribution. In some embodiments, the thickness distribution of the first impedance layer 1130-1 may include the thickness monotonically increasing or monotonically decreasing from a first side of the first impedance layer 1130-1 to a second side, the thickness monotonically increasing or decreasing from a center of the first impedance layer 1130-1 to a periphery, etc. The first side and the second side of the first impedance layer 1130-1 may be different edges of the backing layer 1130, and the first side and the second side may be two edges corresponding to each other. In some embodiments, the direction from the first side of the first impedance layer 1130-1 to the second side may include a width direction of the backing layer 1130 (the Y direction as shown in FIG. 11). In some embodiments, the thickness monotonically increases or monotonically decreases from the first side to the second side of the first impedance layer 1130-1 may include the thickness monotonically increases or monotonically decreases in a stepwise manner, a linear manner, etc. In some embodiments, the thickness monotonically increases or decreases from the center of the first impedance layer 1130-1 to the periphery may include the thickness monotonically increases or decreases in a stepwise manner, a linear manner, a curvilinear manner, a parabolic manner, etc. In some embodiments, the thickness distribution of the above-described first impedance layer 1130-1 may be set according to actual needs and is not limited in the present embodiment.

When the thickness of the piezoelectric layer 1120 is equal at various points, the thickness of the first impedance layer 1130-1 may be unequal to allow for a sound signal that may be emitted by the ultrasonic transducer 100 to remain a signal wave of a range of frequencies. Correspondingly, in some embodiments, the sound velocity of the matching layer 1110 may be in a gradient distribution in at least one direction. The gradient distribution of the sound velocity of the matching layer 1110 allows the ultrasonic waves of different frequencies to have the same wavelength in the matching layer of different fillers. When the thicknesses of the different regions of the matching layer are the same, a ratio of the thicknesses of the different regions to the wavelength of the ultrasonic wave may be close to or reach a desirable value, to improve the transmittance of the acoustic waves of different frequencies and increase the bandwidth of the ultrasonic transducer.

In some embodiments, the gradient distribution of the sound velocity in the matching layer 1110 may be associated with the thickness distribution of the first impedance layer 1130-1. In some embodiments, the gradient distribution of the sound velocity of the matching layer 1110 may be negatively correlated with the thickness distribution of the first impedance layer 1130-1. Exemplarily, in some embodiments, the thickness of the first impedance layer 1130-1 a may monotonically increase from the first side of the first impedance layer 1130-1 to the second side, which may be associated with the sound velocity in the matching layer 1110 monotonically decreasing from the first side to the second side of the matching layer 1110. In some embodiments, the thickness of the first impedance layer 1130-1 monotonically decreasing from the first side of the first impedance layer 1130-1 to the second side may be associated with the sound velocity in the matching layer 1110 monotonically increasing from the first side to the second side of the matching layer 1110. In some embodiments, the thickness of the first impedance layer 1130-1 increasing from the center of the first impedance layer 1130-1 to the periphery may be associated with the sound velocity of the matching layer 1110 decreasing from the center of the matching layer 1110 to the periphery. In some embodiments, the thickness of the first impedance layer 1130-1 decreasing from the center of the first impedance layer 1130-1 to the periphery may be associated with the sound velocity of the matching layer 1110 increasing from the center of the matching layer 1110 to the periphery.

In the embodiment of the present disclosure, the back sound waves may be strongly reflected by setting the impedance layer, so that the ultrasonic transducer may transmit sound signals and also send the reflected back sound waves at the same time, thus increasing the overall strength of the transmitted signals and improving the sensitivity of the transducer. In addition, the thickness distribution of the impedance layer allows for an increase in the frequency range in which the fundamental wave of the sound signal is located, thereby increasing the bandwidth of the ultrasonic transducer. Descriptions regarding the specific implementation of the thickness distribution of the first impedance layer 1130-1 may be found in related descriptions in FIGS. 15-18 and may not be repeated here.

In some embodiments, the thickness distribution of the first impedance layer may be set according to the actual needs and is not limited in the present embodiment. Descriptions regarding the specific implementation of the thickness distribution of the first impedance layer 1130-1 may be found in related descriptions in FIGS. 15-18 and may not be repeated here.

FIG. 12 is a schematic diagram illustrating an exemplary three-dimensional structure of a second type of ultrasonic transducer according to some embodiments of the present disclosure.

As shown in FIG. 12, the second ultrasonic transducer is a broadband array ultrasonic transducer including a matching layer 1110, a piezoelectric layer 1120, a backing layer 1130, and an acoustic lens 1140. The backing layer 1130 includes a first impedance layer 1130-1 and a second impedance layer 1130-2. The piezoelectric layer 1120 may include a first piezoelectric array element 1120-1, a second piezoelectric array element 1120-2, and a third piezoelectric array element 1120-3.

In some embodiments, each structure of the ultrasonic transducer may be connected by bonding. The matching layer 1110, the piezoelectric layer 1120, and the backing layer 1130 may be bonded sequentially to make a second ultrasonic transducer. Exemplarily, a process of obtaining the second ultrasonic transducer is as follows. The piezoelectric layer 1120 and the matching layer 1110 are both of equal thickness at all points, and the upper surface of the piezoelectric layer 1120 is bonded to the lower surface of the matching layer 1110. The backing layer 1130 includes a first impedance layer 1130-1 and a second impedance layer 1130-2. The thickness of the first impedance layer 1130-1 increases from the center of the first impedance layer to the periphery, and the upper surface of the first impedance layer 1130-1 is bonded to the lower surface of the piezoelectric layer 1120. The thickness of the second impedance layer 1130-2 decreases from the center of the second impedance layer to the periphery, the second impedance layer 1130-2 is bonded to the lower surface of the first impedance layer 1130-1, and a lower surface of the second impedance layer 1130-2 is of a planar structure. After the bonding is completed, the bonded second ultrasonic transducer 1100 is obtained as shown in FIG. 11.

FIG. 13 is a schematic diagram illustrating an exemplary cross sectional view in a Y-Z plane of a second type of ultrasonic transducer after bonding and cutting according to some embodiments of the present disclosure.

In some embodiments, the second ultrasonic transducer after bonding may be cut for the first time to obtain the second ultrasonic transducer after being cut. Exemplarily, the second ultrasonic transducer 1300 of three arrays after being cut, as shown in FIG. 13, has a cutting direction along an X-axis direction, and a cutting location may be left and right edges of a middle portion of the first impedance layer 1130-1. In some embodiments, by cutting the bonded second ultrasonic transducer, a second ultrasonic transducer of four arrays, five arrays, etc., may be obtained. In some embodiments, the cutting direction and the cutting location may be set according to the actual needs, which is not limited in the present embodiments.

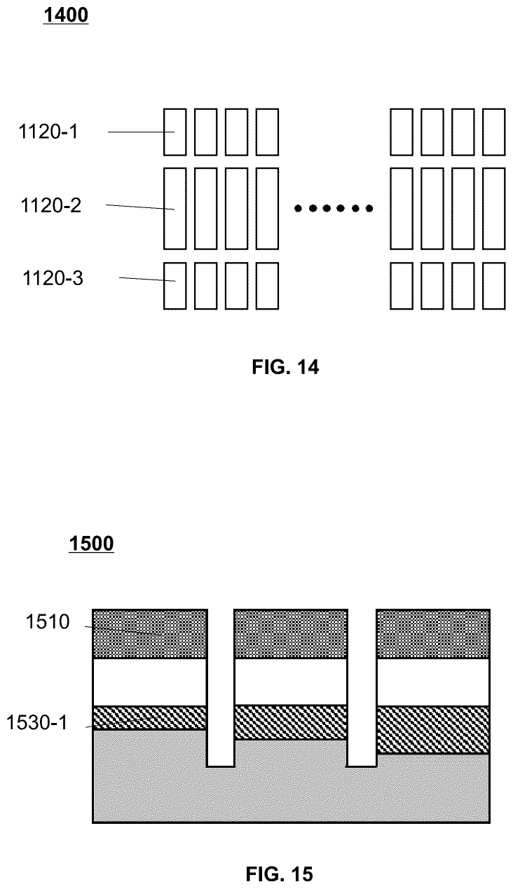

FIG. 14 is a schematic diagram illustrating an exemplary multi-array structure of a second type of ultrasonic transducer after bonding and cutting in an X-Y plane according to some embodiments of the present disclosure.

In some embodiments, the second ultrasonic transducer after bonding and cutting may be cut for the second time to obtain the second ultrasonic transducer after being cut. Exemplarily, the second ultrasonic transducer 1400 of multiple arrays after being cut for a second time, as shown in FIG. 14 has a cutting direction along the Y-axis direction, and a cutting location may be set to be equally spaced. The second ultrasonic transducer having a broadband array may be obtained by bonding an acoustic lens 1140 to the second ultrasonic transducer of multiple arrays after bonding and cutting, such as the second ultrasonic transducer as illustrated in FIG. 12.

In some embodiments of the present disclosure, the third thickness of the first impedance layer 1130-1 (the middle portion of the first impedance layer 1130-1) may be smaller than the fourth thickness (the left portion and the right portion of the first impedance layer 1130-1), and the frequency of ultrasonic waves of the second piezoelectric array element 1120-2 bonding the middle portion of the first impedance layer 1130-1 may be larger than the frequency of ultrasonic waves of the first piezoelectric array element 1120-1 and the third piezoelectric array element 1120-3 boding the left side portion and the right side portion of the first piezoelectric array element 1120-1. Thus, the ultrasonic transducer may satisfy the need for transmitting and receiving ultrasonic waves of different frequencies, the longitudinal resolving power of an image and sensitivity may be improved, and the increase of the bandwidth of the ultrasonic transducer may be favorable to harmonic imaging.

In some embodiments, the piezoelectric layer of the ultrasonic transducer of the broadband array may be a piezoelectric wafer. A piezoelectric array of the piezoelectric layer may be obtained by cutting the piezoelectric wafer of one piezoelectric wafer. The thickness of the piezoelectric layer may be equal at each point, and the piezoelectric layer may have a multi-array parallel structure, which may avoid multiple processing of the piezoelectric wafer and reduce the probability of damage to the piezoelectric wafer.

FIG. 15 is a schematic diagram illustrating an exemplary first type of backing structure of an ultrasonic transducer after bonding according to some embodiments of the present disclosure.

In some embodiments, the sound velocity in the matching layer monotonically decreasing from the first side to the second side may be associated with the thickness of the first impedance layer monotonically increasing from the first side to the second side. As shown in FIG. 15, the gradient distribution of the sound velocity in the matching layer 1510 is associated with the thickness distribution of the first impedance layer 1530-1. The sound velocity of the matching layer 1510 monotonically decreasing from the first side (e.g., the left side) to the second side (e.g., the right side) may be associated with the thickness of the first impedance layer 1530-1 monotonically increasing from the first side (e.g., the left side) to the second side (e.g., the right side) in a stepwise pattern.

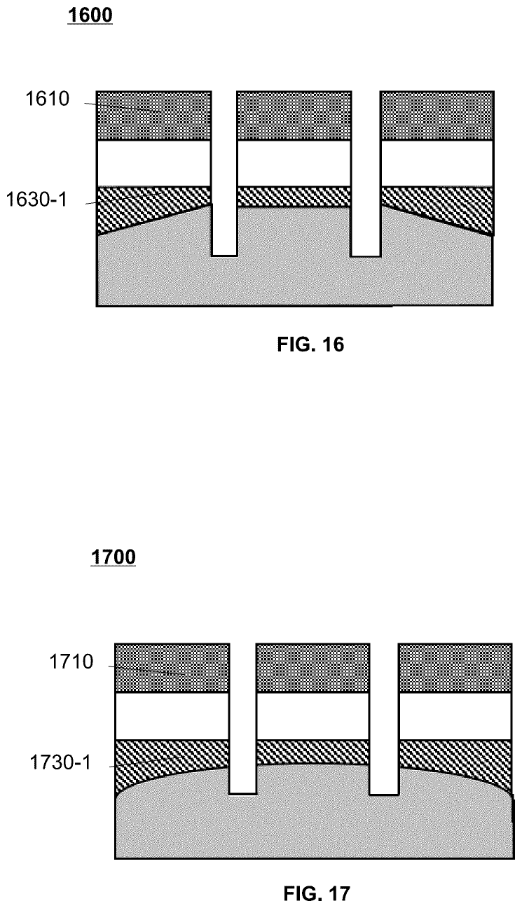

FIG. 16 is a schematic diagram illustrating an exemplary second type of backing structure of an ultrasonic transducer after bonding according to some embodiments of the present disclosure.

In some embodiments, the sound velocity of the matching layer decreasing from the center to the periphery may be associated with the thickness of the first impedance layer increasing from the center to the periphery. As shown in FIG. 16, the gradient distribution of the sound velocity in the matching layer 1610 may be associated with the thickness distribution of the first impedance layer 1630-1. The sound velocity in the matching layer 1610 decreasing (e.g., linearly decreasing) from the center to the periphery may be associated with the thickness of the first impedance layer 1630-1 increasing (e.g., linearly increasing) from the center to the periphery.

FIG. 17 is a schematic diagram illustrating an exemplary third type of backing structure of an ultrasonic transducer after bonding according to some embodiments of the present disclosure.

As shown in FIG. 17, the gradient distribution of the sound velocity in the matching layer 1710 may be associated with the thickness distribution of the first impedance layer 1730-1. The sound velocity in the matching layer 1710 decreasing (e.g., curvilinearly decreasing) from the center to the periphery may be associated with the thickness of the first impedance layer 1730-1 increasing (e.g., curvilinearly increasing) from the center to the periphery.

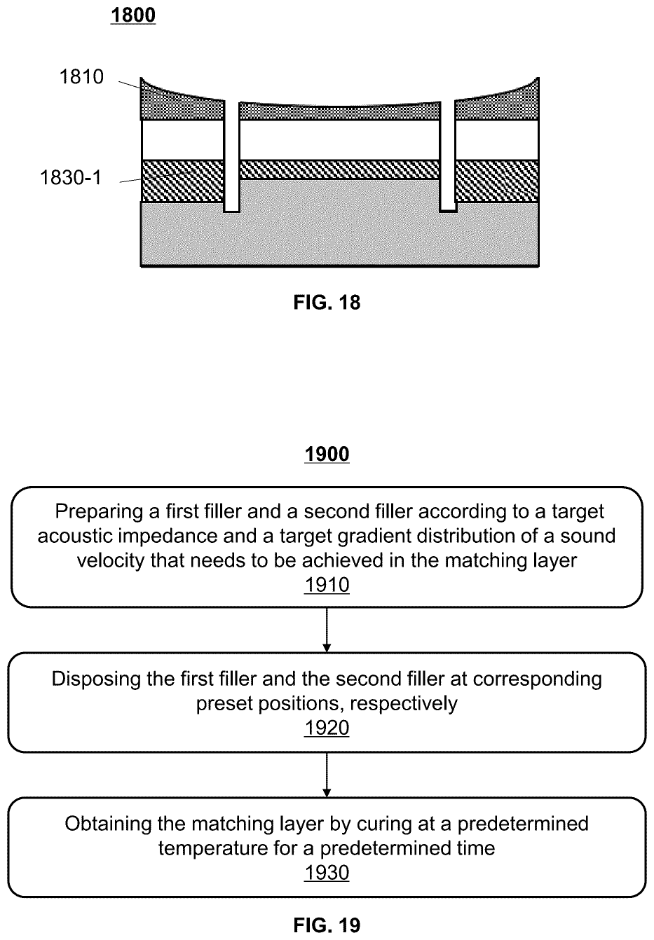

FIG. 18 is a schematic diagram illustrating an exemplary fourth type of backing structure of an ultrasonic transducer after bonding having a matching layer with a non-uniform thickness according to some embodiments of the present disclosure.

As shown in FIG. 18, the thickness of the matching layer 1810 may be different at different points. The gradient distribution of the sound velocity in the matching layer 1810 may be associated with the thickness distribution of the first impedance layer 1830-1. The sound velocity in the matching layer 1810 decreasing from the center to the periphery may be associated with the thickness of the first impedance layer 1830-1 decreasing from the center to the periphery.

FIG. 19 is a flowchart illustrating an exemplary process for preparing a matching layer according to some embodiments of the present disclosure.

As shown in FIG. 19, in some embodiments, the process 1900 may include one or more of the following operations.

In 1910, the first filler and the second filler may be provided with a first filler and a second filler according to a target acoustic impedance and a target gradient distribution of the sound velocity that needs to be achieved in the matching layer.

According to the description of the ultrasonic transducer 100 above, the first filler and the second filler are materials with acoustic properties in the matching layer. In some embodiments, the target acoustic impedance may be the acoustic impedance desired to be achieved by the preparation of the resulting first filler and second filler. In some embodiments, the target acoustic impedance may be designed based on the acoustic impedance of the piezoelectric layer and/or an object to be tested so that the piezoelectric layer may be acoustically matched to the object to be tested by the matching layer. Further, in some embodiments, the first filler and the second filler may also have the same target acoustic impedance to enable acoustic matching between different fillers to reduce the energy consumed by ultrasonic waves when passing through the matching layer, thus improving the transmittance of the ultrasonic waves by the matching layer.

In some embodiments, the target gradient distribution of the sound velocity may be a gradient distribution of the sound velocity desired for the first filler and the second filler obtained by preparation. In some embodiments, the target gradient distribution of the sound velocity may be designed based on a gradient distribution of the thickness of the piezoelectric layer so that ultrasonic waves of different frequencies may have the same wavelength when passing through the matching layer, thereby increasing the transmittance of the matching layer and increasing the bandwidth of the ultrasonic transducer. In some embodiments, the target gradient distribution of the sound velocity may include a desired sound velocity to be achieved by the first filler and the second filler and a desired location at which the first filler and the second filler are to be set (i.e., a preset location described below).

In some embodiments, the distribution of the sound velocity of the first filler and the second filler may be negatively correlated with the thickness distribution of the piezoelectric layer. Exemplarily, when the thickness distribution of the piezoelectric layer is monotonically increasing from the first side to the second side of the piezoelectric layer, the distribution of the sound velocity in the matching layer may also monotonically decrease from the first side (i.e., where the first filler is located) to the second side (i.e., where the second filler is located) of the matching layer. That is, the first filler may be designed to have a greater sound velocity than the second filler.

In some embodiments, the process 1900 may further include providing the first filler and the second filler based on a target density that needs to be achieved for the matching layer. The target density may be a density desired to be achieved by the prepared first filler and the second filler. Because the target acoustic impedance is affected by both the sound velocity and the density, in some embodiments, the target density of the first filler and the target density of the second filler may be designed based on the target gradient distribution of the sound velocity and the target acoustic impedance so that the first filler and the second filler may also have the same target acoustic impedance.

In some embodiments, providing the first filler and the second filler may include providing a type, a percentage, a size, and a structure of a substance in the first filler and the second filler. Exemplarily, both the first filler and the second filler may be configured to include 100 g of epoxy resin, 30 g of epoxy resin curing agent, 1 g of antifoam agent, and 2 g of silane coupling agent KH560 as a base of the first filler and the second filler. Based on the target gradient distribution of the sound velocity and the target sound impedance, one or more following materials may be selected. For example, a material with a sound velocity in a first sound velocity range with a content of less than or equal to 60 g (such as rubber), a material with a sound velocity in the second sound velocity range with a content of less than or equal to 130 g (such as a metal oxide, an inorganic non-metallic compound of a solid structure), a material with a density in the first density range with a content of less than or equal to 500 g (such as metal), and a material with a density in the second density range with a content of less than or equal to 20 g (such as an inorganic non-metallic compound of a hollow structure, a plastic expanding microsphere) to adjust the sound velocity and the acoustic impedance of the first filler and the second filler, so that the matching layer may achieve the target distribution of the sound velocity and the target acoustic impedance.

It should be noted that the first filler and the second filler described above are only as examples, and do not limit the order of magnitude of the filler in the matching layer, but only indicate that there exists a different filler in the matching layer and that the matching layer may have more other fillers, such as a third filler, a fourth filler, etc., which is not specifically limited by the present embodiments.

In 1920, the first filler and the second filler may be provided at a corresponding predetermined position, respectively.

In some embodiments, the predetermined position may be a position where the first filler and the second filler are desired to be set, which may be set based on the target gradient distribution of the sound velocity. In some embodiments, the predetermined position may be configured based on a thickness distribution of the piezoelectric layer. Exemplarily, if the first sound velocity of the first filler is less than the second sound velocity of the second filler, the first filler may be provided in a position corresponding to the piezoelectric layer with a greater thickness, and the second filler may be provided in a position corresponding to the piezoelectric layer with the lesser thickness.

In some embodiments, the first filler and the second filler may be provided at a corresponding predetermined location by pouring, filling, injecting, etc. Exemplarily, the provided first filler and the provided second filler may be potted into the mold in batches, such that the first filler and second filler may be provided at the predetermined position, which enables the gradient distribution of the sound velocity in the matching layer.

In 1930, the matching layer may be obtained by curing at a predetermined temperature for a predetermined time.

In some embodiments, the predetermined temperature may be a temperature required for curing the filler, and the predetermined temperature may be set based on the type, the percentage, and the size of the substance provided in the first filler and the second filler. Exemplarily, if an epoxy resin is selected as the substrate in the first filler and the second filler, a predetermined temperature of 25° C. may be used to cure the filler according to a count of the epoxy resin. In some embodiments, the predetermined time may also be set based on the type, the percentage, and the size of the substance provided in the first filler and the second filler.

In some embodiments, the predetermined temperature may be within a range of 20° C.-100° C., and the predetermined time may be within a range of 2 h-48 h. In some embodiments, a value of the predetermined time may be negatively correlated with a value of the predetermined temperature to ensure that the first filler and the second filler are fully cured. That is, the lower the predetermined temperature, the longer the predetermined time needs to be set, and conversely, the higher the predetermined temperature, the shorter the predetermined time needs to be set. Exemplarily, if the epoxy resin is selected as the substrate in the first filler and the second filler, 100 g of the epoxy resin may be cured using a predetermined temperature of 25° C. and a predetermined time of 24 hours, to prepare and obtain the matching layer. The 100 g of epoxy resin may be cured using a predetermined temperature of 60° C. and a predetermined time of 4 h, thereby preparing to obtain the matching layer. The 100 g of epoxy resin may be cured using a predetermined temperature of 20° C. and a predetermined time of 48 h, thereby preparing to obtain the matching layer.

In some embodiments, the cured matching layer may be affixed and set with the piezoelectric layer by bonding, welding, pinning, etc. In some embodiments, the matching layer may be prepared by molding directly based on the piezoelectric layer, so that the cured matching layer may be set on the upper surface of the piezoelectric layer, thereby realizing the transmission of ultrasonic waves in the ultrasonic transducer. In some embodiments, after the above operation 1930, the matching layer may be ground by a grinder, to quickly and efficiently adjust the thickness of the matching layer.

In some embodiments of the present disclosure, the matching layer obtained by curing the first filler and the second filler may realize the gradient distribution of the sound velocity, so that ultrasonic waves of different frequencies may have the same wavelength in the matching layer, thereby increasing the transmittance of the ultrasonic waves by the matching layer, and increasing the bandwidth of the ultrasonic transducer. Additionally, the matching layer prepared by curing may simplify the process of fabricating the ultrasonic transducer and save costs.

Table 1 shows formulations of a plurality of exemplary fillers and correspondences of acoustic properties. Specific embodiments of the filler in the matching layer are described in detail below in conjunction with Table 1, which illustrates the preparation process of the plurality of exemplary fillers

TABLE 1
Formulation of fillers and correspondence of acoustic properties

| | | Sound | Acoustic | |
|---|---|---|---|---|
| | | velocity | impedance | Density |
| | Formula | (m/s) | (MRayl) | (g/cm³) |
| Filler A | 100 g of epoxy resin + 30 g of curing agent | 2730 | 3.14 | 1.15 |
| Filler B | 100 g of epoxy resin + 30 g of curing agent + 30 g of | 2330 | 2.63 | 1.13 |
| | Nitrile | | | |
| Filler C | 100 g of epoxy resin + 30 g of curing agent + 15 g of | 1917 | 2.07 | 1.08 |
| | silicone rubber A + 15 g of silicone rubber B | | | |
| Filler D | 100 g of epoxy resin + 30 g of curing agent + 30 g of | 1503 | 1.58 | 1.05 |
| | silicone rubber A + 30 g of silicone rubber B | | | |
| Filler E | 100 g of epoxy resin + 30 g of curing agent + 30 g of | 1638 | 3.14 | 1.92 |
| | silicone rubber A + 30 g of silicone rubber B + 150 g of | | | |
| | tungsten | | | |
| Filler F | 100 g of epoxy resin + 30 g of curing agent + 30 g of | 1882 | 3.14 | 1.67 |
| | silicone rubber A + 30 g of silicone rubber B + 118 g of | | | |
| | copper | | | |
| Filler G | 100 g of epoxy resin + 30 g of curing agent + 13 g of | 2960 | 3.14 | 1.06 |
| | 50 μm hollow ceramic microspheres (particle size 30 | | | |
| | μm-70 μm) | | | |
| Filler H | 100 g of epoxy resin + 30 g of curing agent + 130 g of | 3112 | 5.69 | 1.83 |
| | aluminum trioxide microspheres (particle size 5 μm- | | | |
| | 100 μm) | | | |
| Filler I | 100 g of epoxy resin + 30 g of curing agent + 130 g of | 3339 | 6.18 | 1.85 |
| | aluminum trioxide microspheres (particle size 100 μm- | | | |
| | 200 μm) | | | |
| Filler J | 100 g of epoxy resin + 30 g of curing agent + 130 g of | 3143 | 3.14 | 1.00 |
| | aluminum trioxide microspheres (particle size 100 μm- | | | |
| | 200 μm) + 14 g of hollow glass beads (with a density | | | |
| | of 0.15 g/cm³) | | | |
| Filler K | 100 g of epoxy resin + 30 g of curing agent + 65 g of | 3055 | 4.67 | 1.53 |
| | aluminum trioxide microspheres (particle size 5 μm- | | | |
| | 100 μm) | | | |
| Filler L | 100 g of epoxy resin + 30 g of curing agent + 30 g of | 2874 | 3.82 | 1.33 |
| | aluminum trioxide microspheres (particle size 5 μm- | | | |
| | 100 μm) | | | |
| Filler M | 100 g of epoxy resin + 30 g of curing agent + 500 g of | 1719 | 7.64 | 4.45 |
| | tungsten powder | | | |
| Filler N | 100 g of epoxy resin + 30 g of curing agent + 20 g of | 2526 | 1.57 | 0.62 |
| | hollow glass beads (with a density of 0.15 g/cm³) | | | |
| Filler O | 100 g of epoxy resin + 30 g of curing agent + 30 g of | 2791 | 4.97 | 1.78 |
| | nitrile + 130 g of aluminum trioxide microspheres | | | |
| Filler P | 100 g of epoxy resin + 30 g of curing agent + 30 g of | 1816 | 1.80 | 0.99 |
| | silicone rubber A + 30 g of silicone rubber B + 13 g of | | | |
| | hollow ceramic microspheres (particle size 50 μm) | | | |
| Filler Q | Epoxy resin + 30 g of curing agent + 30 g of | 2259 | 3.14 | 1.39 |
| | silicone rubber A + 30 g of silicone rubber B + 50 g of | | | |
| | tungsten + 20 g of aluminum trioxide microspheres | | | |
| Filler R | Epoxy resin + 30 g of curing agent + 30 g of | 2152 | 2.15 | 1.00 |
| | silicone rubber A + 30 g of silicone rubber B + 50 g of | | | |
| | tungsten + 10 g of hollow glass beads (with a density | | | |
| | of 0.15 g/cm³) | | | |
| Filler S | Epoxy resin + 30 g of curing agent + 30 g of silicone | 2427 | 2.45 | 1.01 |
| | rubber A + 30 g of silicone rubber B + 65 g of aluminum | | | |
| | trioxide microspheres + 10 g of hollow glass beads | | | |
| | (with a density of 0.15 g/cm³) | | | |
| Filler T | Epoxy resin + 30 g of curing agent + 30 g of silicone | 2553 | 3.04 | 1.19 |
| | rubber A + 30 g of silicone rubber B + 50 g of tungsten | | | |
| | powder + 65 g of aluminum trioxide microspheres + 10 g | | | |
| | of hollow glass beads (with a density of 0.15 g/cm³) | | | |

### Filler A:

100 g of epoxy resin and 30 g of epoxy resin curing agent were added to a flask and stirred until the mixture was homogeneous (e.g., stirred for 5 minutes, 10 minutes, 15 minutes, etc.). 1 g of antifoam agent (not shown in Table 1) was then added and stirred for a predetermined stirring time (e.g., 3 minutes, 4 minutes, 5 minutes, etc.). Then the epoxy resin was poured into a mold and cured at room temperature for 24 hours to obtain the cured material of the epoxy resin, i.e., the filler A. Acoustic properties of filler A were tested, a sound velocity of filler A was obtained to be 2730 m/s, an acoustic impedance was obtained to be 3.14 MRayl, and a density was obtained to be 1.15 g/cm³.

### Filler B:

100 g of epoxy resin and 30 g of epoxy resin curing agent were added to the flask and stirred until the mixture was homogeneous (e.g., stirred for 5 minutes, 10 minutes, 15 minutes, etc.). 1 g of antifoam agent and 2 g of silane coupling agent KH560 (not shown in Table 1) were then added and stirred for a predetermined stirring time (e.g., 3 minutes, 4 minutes, 5 minutes, etc.). 30 g of liquid nitrile rubber was added to the flask and stirred until the mixture was homogeneous (e.g., stirred for 5 minutes, 10 minutes, 15 minutes, etc.). Then the co-blended resin was poured into the mold, and cured at room temperature for 24 hours to obtain a modified cured material of the epoxy resin of the nitrile rubber, i.e., filler B. Acoustic properties of filler B were tested, and a sound velocity of filler B was obtained to be 2130 m/s, the acoustic impedance was obtained to be 2.63 MRayl, and the density was obtained to be 1.13 g/cm³.

### Filler C:

100 g of epoxy resin and 30 g of epoxy resin curing agent were added to the flask and stirred until the mixture was homogeneous (e.g., stirred for 5 minutes, 10 minutes, 15 minutes, etc.). 1 g of antifoam agent and 2 g of silane coupling agent KH560 (not shown in Table 1) were then added and stirred for the predetermined stirring time (e.g., 3 minutes, 4 minutes, 5 minutes, etc.). 15 g of silicone rubber A and 15 g of silicone rubber B were added to the flask and stirred until the mixture was homogeneous (e.g., stirred for 5 minutes, 10 minutes, 15 minutes, etc.). Then the co-blended resin was poured into the mold, and cured at room temperature for 24 hours to obtain a modified cured material of the epoxy resin of the silicone rubber, i.e., the filler C. Acoustic properties of filler C were tested, and a sound velocity of filler C was obtained to be 1917 m/s, an acoustic impedance was obtained to be 2.07 MRayl, and a density was obtained to be 1.08 g/cm³.

From the above filler A, filler B, and filler C, it may be seen that the sound velocity of the filler B and the sound velocity of the filler C may be less than the sound velocity of the filler A, and the acoustic impedance of the filler B and the acoustic impedance of the filler C may be less than the acoustic impedance of the filler A. That is to say, due to the characteristic of low sound velocity of the rubber filler, adding rubber to the epoxy resin may reduce the sound velocity of the epoxy resin. Compared to the epoxy resin (i.e., the filler A), a modified epoxy resin (i.e., the filler B and the filler C) with reduced sound velocity may be obtained by introducing rubber, but at the same time, the acoustic impedance of the modified epoxy resin may be reduced.

### Filler D:

100 g of epoxy resin and 30 g of epoxy resin curing agent were added to the flask and stirred until the mixture was homogeneous (e.g., stirred for 5 minutes, 10 minutes, 15 minutes, etc.). 1 g of antifoam agent and 2 g of silane coupling agent KH560 were then added and stirred for the predetermined stirring time (e.g., 3 minutes, 4 minutes, 5 minutes, etc.). 30 g of silicone rubber A and 30 g of silicone rubber B were added to the flask and stirred until the mixture was homogeneous (e.g., stirred for 5 minutes, 10 minutes, 15 minutes, etc.). Then the co-blended resins were poured into the mold, and cured at room temperature for 24 hours to obtain a modified cured material of the epoxy resin of the silicone rubber, i.e., filler D. Acoustic property of filler D was tested, and a sound velocity of filler D was obtained to be 1503 m/s, the acoustic impedance was obtained to be 1.58 MRayl, and the density was obtained to be 1.05 g/cm³.

From the above filler A, filler C, and filler D, it may be seen that the sound velocity and the acoustic impedance of filler D are smaller than that of filler C. That is, compared to the epoxy resin (i.e., filler A), the sound velocity and acoustic impedance of the modified curing material of the epoxy resin may be further reduced with the increase in the amount of rubber filler added.

### Filler E:

100 g of epoxy resin and 30 g of epoxy resin curing agent were added to the flask and stirred until the mixture was homogeneous (e.g., stirred for 5 minutes, 10 minutes, 15 minutes, etc.). 1 g of antifoam agent and 2 g of silane coupling agent KH560 were then added and stirred for the predetermined stirring time (e.g., 3 minutes, 4 minutes, 5 minutes, etc.). A total of 150 g of tungsten powder with a particle size of 3 μm was added to the flask two times in 10 minutes. 30 g of silicone rubber A and 30 g of silicone rubber B were added to the flask and stirred until the mixture was homogeneous (e.g., stirred for 5 minutes, 10 minutes, 15 minutes, etc.). Then the co-blended resin was poured into the mold, and cured at room temperature for 24 hours to obtain a modified cured material of the epoxy resin, i.e., the filler E. Acoustic properties of filler E were tested, and a sound velocity of filler E was obtained to be 1638 m/s, an acoustic impedance of filler E was obtained to be 3.14 MRayl, and a density of filler E was obtained to be 1.92 g/cm³.

From the above filler A, filler D, and filler E, it may be seen that the sound velocity of filler D and filler E is less than the sound velocity of filler A, the density of filler D is less than that of filler E, and filler A and filler E have the same acoustic impedance. That is, the addition of rubber to the epoxy resin (i.e., filler A) may reduce the sound velocity, but the acoustic impedance of the modified epoxy curing material (i.e., filler D) may be reduced. Based on the modified curing material of the epoxy resin (i.e., filler D), a high-density tungsten powder may be introduced to adjust the property of the filler, and a modified epoxy resin (i.e., filler E) with improved density may be obtained, resulting in an improvement in the acoustic impedance of the modified epoxy resin (i.e., filler E). While the acoustic impedance of the modified epoxy resin (i.e., filler A) with improved density (i.e., filler E) was the same as that of the epoxy resin (i.e., filler A) compared to the modified epoxy resin (i.e., filler E), the sound velocity of the modified epoxy resin (i.e., filler E) may be lower.

### Filler F:

100 g of epoxy resin and 30 g of epoxy resin curing agent were added to the flask and stirred until the mixture is homogeneous (e.g., stirred for 5 minutes, 10 minutes, 15 minutes, etc.). Then 1 g of antifoam agent and 2 g of silane coupling agent KH560 were then added and stirred for the predetermined stirring time (e.g., stirred for 3 minutes, 4 minutes, 5 minutes, etc.). A total of 118 g of copper powder with a particle size of 20 μm was added to the flask two times in 10 minutes. 30 g of silicone rubber A and 30 g of silicone rubber B were added to the flask and stirred until the mixture was homogeneous (e.g., stirred for 5 minutes, 10 minutes, 15 minutes, etc.). Then the co-blended resin was poured into the mold, and cured at room temperature for 24 hours to obtain a modified cured material of the epoxy resin, i.e., the filler F. Acoustic properties of filler F were tested, and a sound velocity of filler F was obtained to be 1882 m/s, an acoustic impedance of filler F was obtained to be 3.14 MRayl, and a density of filler F was obtained to be 1.67 g/cm³.

From the above filler D, filler E, and filler F, it may be seen that the acoustic impedance of filler E and filler F is greater than that of filler D, and the sound velocity of filler F is greater than the sound velocity of filler D and filler E. In other words, both tungsten powder and copper powder increase the acoustic impedance of the modified epoxy resin (i.e., filler D). However, due to the low density of the copper powder and the large volume fraction of the added copper powder, the addition of the copper powder enhances the sound velocity at a higher level compared to the addition of tungsten powder.

### Filler G:

100 g of epoxy resin and 30 g of epoxy resin curing agent were added to the flask and stirred until the mixture was homogeneous (e.g., stirred for 5 minutes, 10 minutes, 15 minutes, etc.). Then 1 g of antifoam agent and 2 g of KH560 were added to the flask and stirred for the predetermined stirring time (e.g., 3 minutes, 4 minutes, 5 minutes, etc.). A total of 13 g of hollow ceramic microspheres with a particle size of 50 μm were added to the flask two times and mixed evenly in 10 minutes. Then the modified epoxy resin was added to the mold, and cured at room temperature for 24 hours to obtain a modified cured material of the epoxy resin, i.e., the filler G. Acoustic properties of filler G were tested, and a sound velocity of filler G was obtained to be 2960 m/s, an acoustic impedance of filler G was obtained to be 3.14 MRayl, and a density of filler G was obtained to be 1.06 g/cm³.

From filler A to filler G, it may be seen that the sound velocity of filler G is greater than that of filler A, the density of filler G is less than that of filler A, the acoustic impedance of filler G is the same as that of filler A, and the acoustic resistance of filler G is the same as that of filler A. That is, by adding the hollow ceramic microspheres to the epoxy resin (i.e., filler A), the sound velocity may be increased, and the impedance may remain unchanged. The addition of the hollow ceramic microspheres on the one hand may increase the sound velocity, and on the other hand may reduce the density, so that the sound velocity may be increased, and the acoustic impedance may remain unchanged.

### Filler H:

100 g of epoxy resin and 30 g of epoxy resin curing agent were added to the flask and stirred until the mixture was homogeneous (e.g., stirred for 5 minutes, 10 minutes, 15 minutes, etc.). 1 g of antifoam agent and 2 g of silane coupling agent KH560 were then added and stirred for the predetermined stirring time (e.g., 3 minutes, 4 minutes, 5 minutes, etc.). A total of 40 g of aluminum trioxide microspheres with a particle size of 50 μm were added to the flask two times in 10 minutes. The modified epoxy resin was added to the mold after the mixture was homogeneous, and cured at room temperature for 24 hours to obtain a modified cured material of the epoxy resin, i.e., the filler H. Acoustic properties of filler H were tested, and a sound velocity of filler H was obtained to be 3112 m/s, an acoustic impedance of filler H was obtained to be 5.69 MRayl, and a density of filler H was obtained to be 1.83 g/cm³.

### Filler I:

100 g of epoxy resin and 30 g of epoxy resin curing agent were added to the flask and stirred until the mixture was homogeneous (e.g., stirred for 5 minutes, 10 minutes, 15 minutes, etc.). 1 g of antifoam agent and 2 g of silane coupling agent KH560 were then added and stirred for the predetermined stirring time (e.g., 3 minutes, 4 minutes, 5 minutes, etc.). A total of 40 g of aluminum trioxide microspheres with a particle size of 150 μm were added to the flask two times in 10 minutes. The modified epoxy resin was added to the mold after the mixture was homogeneous, and cured at room temperature for 24 hours to obtain a modified cured material of the epoxy resin, i.e., the filler I. Acoustic properties of filler I were tested, and a sound velocity of filler I was obtained to be 3339 m/s, an acoustic impedance of filler H was obtained to be 6.18 MRayl, and a density of filler I was obtained to be 1.853 g/cm³.

### Filler J:

100 g of epoxy resin and 30 g of epoxy resin curing agent were added to the flask and stirred until the mixture was homogeneous. 1 g of antifoam agent and 2 g of silane coupling agent KH560 were then added and stirred for the predetermined stirring time (e.g., 3 minutes, 4 minutes, 5 minutes, etc.). A total of 40 g of aluminum trioxide microspheres and hollow glass beads with a density of 0.15 g/cm³ were added to the flask two times in 10 minutes. The modified epoxy resin was added to the mold after the mixture was homogeneous, and cured at room temperature for 24 hours to obtain a modified cured material of the epoxy resin, i.e., the filler J. Acoustic properties of filler J were tested, and a sound velocity of filler J was obtained to be 3143 m/s, an acoustic impedance of filler J was obtained to be 3.14 MRayl, and a density of filler J was obtained to be 1.00 g/cm³.

From filler H to filler J, it may be seen that as the particle size of the microspheres increases, the sound velocity is increased. Then by adding low-density hollow glass beads, the modified epoxy resin (i.e., filler A) with an impedance of 3.14 Mrayl (the same acoustic impedance as that of filler A), and a high sound velocity may be obtained compared to the epoxy resin (i.e., filler J).

### Filler K-Filler L:

The filler K includes 100 g of epoxy resin, 30 g of epoxy resin curing agent, 1 g of antifoam agent, 2 g of silane coupling agent KH560, and 65 g of aluminum trioxide microspheres with a particle size of 5 μm-100 μm. Acoustic properties of the filler K were tested, and a sound velocity of the filler K was obtained to be 3055 m/s, an acoustic impedance of the filler K was obtained to be 4.67 MRayl, and a density of the filler K was obtained to be 1.53 g/cm³.

Filler L includes 100 g of epoxy resin, 30 g of epoxy resin curing agent, 1 g of antifoam agent, 2 g of silane coupling agent KH560, and 30 g of aluminum trioxide microspheres with a particle size of 5 μm-100 μm. Acoustic properties of the filler L were tested, and a sound velocity of the filler L was obtained to be 2874 m/s, an acoustic impedance of the filler L was obtained to be 3.82 MRayl, and a density of the filler L was obtained to be 1.33 g/cm³. Filler K and filler L are prepared similarly as filler H to filler I described above and may not be repeated here.

From filler H to filler I and filler K to filler L, it is known that as the content of metal oxides in the filler increases, the sound velocity of the filler increases, and the metal oxides in the first filler as well as in the second filler may be adjusted so that the first filler and the second filler have different sound velocities.

The formulations and acoustic properties of several exemplary fillers are also provided below, which are prepared similarly as the filler A to filler J described above and are not repeated herein.

### Filler M:

The filler M includes 100 g of epoxy resin, 30 g of epoxy resin curing agent, antifoam agent, 2 g of silane coupling agent KH560, and 500 g of tungsten powder. Acoustic properties of the filler M were tested, and a sound velocity of the filler M was obtained to be 1719 m/s, an acoustic impedance of the filler M was obtained to be 7.64 MRayl, and a density of the filler M was obtained to be 4.45 g/cm³. From filler A and filler M, it may be seen that the separate addition of tungsten powder into the epoxy resin may dramatically increase the acoustic impedance and density of the epoxy resin and reduce the sound velocity of the epoxy resin.

### Filler N:

The filler N includes 100 g of epoxy resin, 30 g of epoxy resin curing agent, 1 g of antifoam agent, 2 g of silane coupling agent KH560, and 20 g of hollow glass beads with a density of 0.15 g/cm³. Acoustic properties of the filler N were tested, and a sound velocity of the filler N was obtained to be 2526 m/s, an acoustic impedance of the filler N was obtained to be 1.57 MRayl, and a density of the filler N was obtained to be 0.62 g/cm³. From filler A and filler N, it may be seen that the separate addition of hollow glass beads into the epoxy resin may dramatically reduce the density of the epoxy resin and decrease the sound velocity and acoustic impedance of the epoxy resin.

### Filler O:

The filler O includes 100 g of epoxy resin, 30 g of epoxy resin curing agent, 1 g of defoamer, 2 g of silane coupling agent KH560, 30 g of nitrile, and 130 g of aluminum trioxide microspheres. Acoustic properties of the filler O were tested, and a sound velocity of the filler O was obtained to be 2791 m/s, an acoustic impedance of the filler O was obtained to be 4.97 MRayl, and a density of the filler O was obtained to be 1.78 g/cm³.

### Filler P:

The filler P includes 100 g of epoxy resin, 30 g of epoxy resin curing agent, 1 g of antifoam agent, 2 g of silane coupling agent KH560, 30 g of silicone rubber A, 30 g of silicone rubber B, and 13 g of hollow ceramic microspheres with a particle size of 50 μm. Acoustic properties of the filler P were tested, and a sound velocity of the filler P was obtained to be 1816 m/s, an acoustic impedance of the filler P was obtained to be 1.80 MRayl, and a density of the filler P was obtained to be 0.99 g/cm³.

### Filler Q:

Filler Q includes 100 g of epoxy resin, 30 g of epoxy resin curing agent, 1 g of antifoam agent, 2 g of silane coupling agent KH560, 30 g of silicone rubber A, 30 g of silicone rubber B, 50 g of tungsten, and 20 g of aluminum trioxide microspheres. Acoustic properties of the filler Q were tested, and a sound velocity of the filler Q was obtained to be 2259 m/s, the acoustic impedance of the filler Q was obtained to be 3.14 MRayl, and a density of the filler Q was obtained to be 1.39 g/cm³.

### Filler R:

The filler R includes 100 g of epoxy resin, 30 g of epoxy resin curing agent, 1 g of antifoam agent, 2 g of silane coupling agent KH560, 30 g of silicone rubber A, 30 g of silicone rubber B, 50 g of tungsten, and 10 g of hollow glass beads with a density of 0.15 g/cm³. Acoustic properties of the filler R were tested, and a sound velocity of the filler R was obtained to be 2152 m/s, an acoustic impedance of the filler R was obtained to be 2.15 MRayl, and a density of the filler R was obtained to be 1.00 g/cm³.

### Filler S:

The filler S includes 100 g of epoxy resin, 30 g of epoxy resin curing agent, 1 g of antifoam agent, 2 g of silane coupling agent KH560, 30 g of silicone rubber A, 30 g of silicone rubber B, 65 g of aluminum trioxide microspheres, and 10 g of hollow glass beads with a density of 0.15 g/cm³. Acoustic properties of the filler S were tested, and a sound velocity of the filler S was obtained to be 2427 m/s, an acoustic impedance of the filler S was obtained to be 2.45 MRayl, and a density of the filler S was obtained to be 1.01 g/cm³.

### Filler T:

The filler T includes 100 g of epoxy resin, 30 g of epoxy resin curing agent, 1 g of antifoam agent, 2 g of silane coupling agent KH560, 30 g of silicone rubber A, 30 g of silicone rubber B, 50 g of tungsten powder, 65 g of alumina trioxide microspheres, and 10 g of hollow glass beads with a density of 0.15 g/cm³. Acoustic properties of the filler T were tested, and a sound velocity of the filler T was obtained to be 2553 m/s, an acoustic impedance of the filler T was obtained to be 3.04 MRayl, and a density of the filler T was obtained to be 1.19 g/cm³.

In some embodiments, one or more combinations of one or more of the filler B to filler T may be selected as the first filler and the second filler to enable the first filler and the second filler to have different sound velocities to achieve a target distribution of the sound velocity in the matching layer. Further, in some embodiments, one or more combinations of one or more of filler E, filler F, filler G, filler J, and filler Q may be selected as the first filler and the second filler to enable the first filler and the second filler to have different sound velocities as well as the same acoustic impedance to achieve a target distribution of the sound velocity and a target acoustic impedance in the matching layer.

It should be noted that the acoustic property parameters of the fillers in Table 1 are only for illustrative purposes, and the present disclosure does not specifically limit the acoustic property parameters of the fillers in Table 1 herein.

Beneficial effects that may be brought about by embodiments of the present disclosure include but are not limited to the following. Through the gradient distribution of the sound velocity in the matching layer, ultrasonic waves with different frequencies have the same wavelength in the matching layer of different structures, thereby realizing acoustic matching between the piezoelectric layer of different structures and human body tissues, improving the transmittance of the ultrasonic waves in the matching layer, and increasing the bandwidth of the ultrasonic transducer. By filling fillers with different sound velocities at different locations, the sound velocities in the formed matching layer are made to have a gradient distribution in at least one direction (e.g., an elevation direction of the matching layer), thereby increasing the transmittance of the matching layer to ultrasonic waves of different frequencies and increasing the bandwidth of the ultrasonic transducer.

Having thus described the basic concepts, it may be rather apparent to those skilled in the art after reading this detailed disclosure that the foregoing detailed disclosure is intended to be presented by way of example only and is not limiting.

Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims.

Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the invention. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

1. An ultrasonic transducer, comprising a piezoelectric layer and a matching layer, wherein:
the matching layer is provided between the piezoelectric layer and an object to be measured, the piezoelectric layer being acoustically matched with the object through the matching layer, and the piezoelectric layer being configured to facilitate conversion between ultrasonic waves and electrical energy; and
a sound velocity in the matching layer is in a gradient distribution in at least one direction.

2. The ultrasonic transducer of claim 1, wherein
a thickness of the piezoelectric layer includes a first thickness and a second thickness; and
the first thickness is unequal to the second thickness.

3. The ultrasonic transducer of claim 2, wherein the gradient distribution of the sound velocity in the matching layer is associated with a thickness distribution of the piezoelectric layer.

4. The ultrasonic transducer of claim 3, wherein
the gradient distribution of the sound velocity in the matching layer is negatively correlated with the thickness distribution of the piezoelectric layer.

5. The ultrasonic transducer of claim 1, further comprising a backing layer, wherein:
the backing layer includes a first impedance layer and a second impedance layer;
the first impedance layer is connected to the second impedance layer;
the first impedance layer is connected to a surface of the piezoelectric layer away from the matching layer; and
an impedance of the first impedance layer is higher than an impedance of the second impedance layer.

6. The ultrasonic transducer of claim 5, wherein
the piezoelectric layer is of equal thickness at different sites;
a thickness of the first impedance layer includes a third thickness and a fourth thickness; and
the third thickness is unequal to the fourth thickness.

7. The ultrasonic transducer of claim 5, wherein the gradient distribution of the sound velocity in the matching layer is associated with a thickness distribution of the first impedance layer.

8. The ultrasonic transducer of claim 7, wherein:
the gradient distribution of the sound velocity in the matching layer is negatively correlated with the thickness distribution of the first impedance layer; and/or
the gradient distribution of the sound velocity in the matching layer is negatively correlated with a thickness distribution of a thickness sum of the first impedance layer and the piezoelectric layer.

9. The ultrasonic transducer of claim 1, wherein the matching layer is of equal thickness at different sites.

10. The ultrasonic transducer of claim 1, wherein
a thickness of the matching layer includes a fifth thickness and a sixth thickness; and
the fifth thickness is unequal to the sixth thickness.

11. The ultrasonic transducer of claim 1, wherein:
the piezoelectric layer includes one or more piezoelectric array elements; and
the at least one direction includes a length direction and/or a thickness direction of the one or more piezoelectric array elements.

12. The ultrasonic transducer of claim 1, wherein:
the matching layer includes a first filler and a second filler; and
a first sound velocity corresponding to the first filler is different from a second sound velocity corresponding to the second filler.

13. The ultrasonic transducer of claim 12, wherein the first filler and the second filler are respectively filled at different positions in a same plane of the matching layer to achieve a gradient distribution of the sound velocity in the matching layer.

14. The ultrasonic transducer of claim 12, wherein acoustic impedances and/or thicknesses at positions of the matching layer at which the first filler and the second filler are respectively filled, are the same.

15. The ultrasonic transducer of claim 12, wherein:
the first filler includes at least one type of material that is different from the second filler; or
the first filler and the second filler include one or more same types of materials, wherein percentages, sizes, and/or structures of at least one of the one or more same types of materials in the first filler and the second filler are different.

16. (canceled)

17. The ultrasonic transducer of claim 12, wherein
the first filler and/or the second filler include an epoxy resin; and
the first filler and/or the second filler further include at least one of a material with a sound velocity in a first sound velocity range, a material with a sound velocity in a second sound velocity range, a material with a density in a first density range, and a material with a density in a second density range, the first sound velocity range being smaller than the second sound velocity range, and the first density range being greater than the second density range.

18-19. (canceled)

20. The ultrasonic transducer of claim 17, wherein:
the material with the sound velocity in the first sound velocity range includes rubber;
the material with the sound velocity in the second sound velocity range includes a metal oxide and/or an inorganic non-metallic compound of a solid structure;
the material with the density in the first density range includes a metal; and
the material with the density in the second density range includes an inorganic non-metallic compound of a hollow structure and/or a plastic expanding microsphere.

21. The ultrasonic transducer of claim 20, wherein
materials of the first filler include the epoxy resin, the rubber, and the metal;
materials of the second filler include the epoxy resin, the metal oxide, and the inorganic non-metallic compound of hollow structure;
a first sound velocity of the first filler is less than a second sound velocity of the second filler; and
an acoustic impedance of the first filler and an acoustic impedance of the second filler are the same.

22. The ultrasonic transducer of claim 20, wherein
the epoxy resin includes at least one of an epoxy resin of a bisphenol A type or an epoxy resin of a bisphenol F type;
the rubber includes at least one of thermoplastic SBS elastomer, nitrile rubber, a butyl rubber, styrene-butadiene rubber, male-butadiene rubber, ethylene propylene diene monomer (EPDM) rubber, silicone rubber, or fluorine rubber;
the metal includes at least one of tungsten, copper, iron, or lead;
the metal oxide includes at least one of a tungsten trioxide, an iron oxide, an aluminum trioxide, a zinc oxide, or a magnesium oxide; and
the inorganic non-metallic compound includes at least one of glass, ceramic, or boron carbide.

23-24. (canceled)

25. A method for preparing a matching layer, wherein the matching layer is applied in an ultrasonic transducer, the ultrasonic transducer including a piezoelectric layer and a matching layer, the matching layer being provided between the piezoelectric layer and an object to be measured, the piezoelectric layer being acoustically matched with the object through the matching layer, the piezoelectric layer being configured to facilitate conversion between ultrasonic waves and electrical energy, and a sound velocity in the matching layer being in a gradient distribution in at least one direction, and the method comprises:
preparing a first filler and a second filler according to a target acoustic impedance and a target gradient distribution of a sound velocity that need to be achieved in the matching layer;
disposing the first filler and the second filler in corresponding predetermined positions, respectively; and
obtaining the matching layer by curing at a predetermined temperature for a predetermined time.

26. (canceled)
